(19) United States
(12) Patent Application Publication
(10) Pub. No.: US 2004/0180901 A1
(43) Pub. Date: Sep. 16, 2004

(54) Arylpiperazines and arylpiperidines and their use as metalloproteinase inhibiting agents

(76) Inventors: Raymond Finlay, Macclesfield (GB); David Waterson, Macclesfield (GB)

Correspondence Address: Ropes & Gray, One International Place, Boston, MA, 02110-2624, US

(21) Appl. No.: 10/485,675

(22) PCT Filed: Aug. 8, 2002
(86) PCT No.: PCT/SE02/01437

(30) Foreign Application Priority Data
Aug. 9, 2001 (GB) 0119472.9

(51) Int. Cl. A61K 31/495; A61K 31/496; A61K 31/445

(52) U.S. Cl. 514/253.01; 514/255.02; 544/360; 544/383; 514/318; 514/317

(57) ABSTRACT

Compounds of the formula (I) useful as metalloproteinase inhibitors, especially as inhibitors of MMP 13.

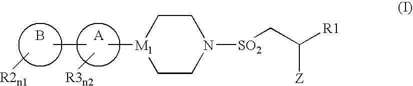

### CROSS-REFERENCE TO RELATED APPLICATIONS

This application is a national stage filing under 35 U.S.C. 371 of International Application No. PCT/SE02/01437, filed Aug. 8, 2002, which claims priority from United Kingdom Patent Application No. 0119472.9, filed Aug. 9, 2001, the specification of which is incorporated by reference herein. International Application No. PCT/SE02/01038 was published under PCT Article 21(2) in English.

### FIELD OF THE INVENTION

The present invention relates to compounds useful in the inhibition of metalloproteinases and in particular to pharmaceutical compositions comprising these, as well as their use. In particular, the compounds of this invention are inhibitors of matrix metalloproteinase 13 (MMP13), known also as collagenase 3.

Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMPs); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), comeal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; and chronic obstructive pulmonary diseases, COPD (for example, the role of MMPs such as MMP12 is discussed in Anderson & Shinagawa, 1999, Current Opinion in Anti-inflammatory and Immunomodulatory Investigational Drugs, 1(1): 29-38).

The matrix metalloproteinases (MMPs) are a family of structurally-related zinc-containing endopeptidases which mediate the breakdown of connective tissue macro-molecules. The mammalian MMP family is composed of at least twenty enzymes, classically divided into four sub-groups based on substrate specificity and domain structure [Alexander & Werb (1991) in Hay, E. D. ed. “Cell Biology of the Extracellular Matrix”, New York, Plenum Press, 255-302; Murphy & Reynolds (1993) in Royce, P. M. & Steinman, B. eds. “Connective Tissue and its Heritable Disorders”, New York, Wiley-Liss Inc., 287-316; Birkedal-Hansen (1995) Curr. Opin. Cell Biol. 7:728-735]. The sub-groups are the collagenases (such as MMP1, MMP8, MMP13), the stromelysins (such as MMP3, MMP10, MMP11), the gelatinases (such as MMP2, MMP9) and the membrane-type MMPs (such as MMP14, MMP15, MMP16, MMP17). Enzyme activity is normally regulated in vivo by tissue inhibitors of metalloproteinases (TIMPs).

Because of their central role in re-modelling connective tissue, both as part of normal physiological growth and repair and as part of disease processes, there has been substantial interest in these proteins as targets for therapeutic intervention in a wide range of degenerative and inflammatory diseases, such as arthritis, atherosclerosis, and cancer [Whittaker et al (1999) Chem. Rev. 99:2735-2776).

A number of MMP inhibitor compounds are known and some are being developed for pharmaceutical uses (see for example the review by Beckett & Whittaker (1998) Exp. Opin. Ther. Patents, 8(3):259-282). Different classes of compounds may have different degrees of potency and selectivity for inhibiting various MMPs. Whittaker M. et al (1999, Chem. Rev. 99:2735-2776) review a wide range of known MMP inhibitor compounds. They state that an effective MMP inhibitor requires a zinc binding group or ZBG (functional group capable of chelating the active site zinc(II) ion), at least one functional group which provides a hydrogen bond interaction with the enzyme backbone, and one or more side chains which undergo effective van der Waals interactions with the enzyme subsites. Zinc binding groups in known MMP inhibitors include hydroxamic acids (—C(O)NHOH), reverse hydroxamates (—N(OH)CHO), thiols, carboxylates and phosphonic acids.

We have discovered a new class of compounds that are inhibitors of metalloproteinases and are of particular interest in inhibiting MMP13. The compounds of this invention have beneficial potency and/or pharmacokinetic properties. In particular they show selectivity for MMP13.

MMP13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP13 plays a role in the turnover of other connective tissues. For instance, consistent with MMP13's substrate specificity and preference for degrading type II collagen [P. G. Mitchell et al., (1996) J. Clin. Invest. 97(3):761-768; V. Knauper et al., (1996) The Biochemical Journal 271:1544-1550], MMP13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wemicke et al., (1996) J. Rheumatol. 23:590-595; P. G. Mitchell et al., (1996) J. Clin. Invest. 97(3):761-768; 0. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6):1489-1499] and in remodelling of the collagenous matrix in chronic wounds [M. Vaalamo et al., (1997) J. Invest. Dermatol. 109(1):96-101].

U.S. Pat. No. 6,100,266 and WO-99/38843 disclose compounds of the general formula

B—X—(CH₂)ₘ—CR¹R²)ₙ—W—COY

for use in the manufacture of a medicament for the treatment or prevention of a condition associated with matrix metalloproteinases. Specifically disclosed is the compound N-{1S-[4-(4-Chlorophenyl)piperazine-1-sulfonylmethyl]-2-methylpropyl}-N-hydroxyformamide.

WO-01/87870 discloses hydroxamic acid derivatives of the general formula

D-B—X-A-SO₂—CH₂—(CR²R³)—CONHOH

wherein D and B are each an aryl or heteroaryl ring and A is a heterocyclic ring, for use as inhibitors of matrix metalloproteinases.

WO-00/12478 discloses arylpiperazines that are matrix metalloproteinase inhibitors, including compounds with an hydroxamic acid zinc binding group and compounds with a reverse hydroxamate zinc binding group.

WO-2000/51993 claims dihetero-substituted metalloprotease ihibitors, including a compound of the formula:

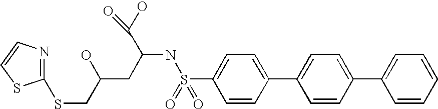

We have now discovered compounds that are potent MMP13 inhibitors and have desirable activity profiles.

In a first aspect of the invention we now provide a compound of the formula I

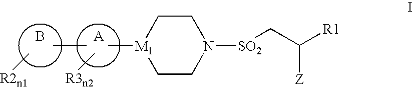

wherein

A and B are each independently selected from phenyl and up to C6 heteroaryl;

at least one of A and B is heteroaryl;

n1 and n2 are each independently selected from 0, 1, 2, 3;

each R2 and each R3 is independently selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy;

M₁ is selected from N and C;

R1 is the group —X—Y;

X is C₁₋₆alkyl;

Y is selected from up to C₁₀ cycloalkyl, up to C₁₀ aryl, and up to C₁₀ heteroaryl;

Y is optionally substituted by up to three groups independently selected from OH, NO₂, CF₃, CN, halogen, SC 4alkyl, SOC₁₋₄alkyl, SO₂C₁₋₄alkyl, C₁₋₄alkyl, C₁₋₄alkoxy;

Z is selected from —N(OH)CHO, and —C(O)NHOH;

Any heteroaryl group outlined above is an aromatic ring containing one or more heteroatoms independently selected from N, O, S;

Any alkyl group outlined above may be straight chain or branched.

Preferred compounds of the formula I are those wherein any one or more of the following apply:

at least one of A and B is a five- or six-membered aromatic ring containing one or more heteroatoms independently selected from N, O, S; preferably at least one of A and B is pyridyl, pyrimidinyl, thienyl, furyl;

B is not substituted or is substituted by at least one R2 group selected from CF₃, CN, halogen (preferably fluoro or chloro), C₁₋₄alkyl;

A is not substituted or is substituted by at least one R3 group selected from CF₃, CN, halogen (preferably fluoro or chloro), C₁₋₄alkyl;

M₁ is N;

X is C₂₋₅alkyl; preferably X is C₂₋₃alkyl;

Y is selected from phenyl and a five- or six-membered aromatic ring containing one or more heteroatoms independently selected from N, O, S; preferably Y is phenyl, pyridyl, pyrimidinyl, or pyrazinyl; most preferably Y is pyrimidinyl;

Y is not substituted or is substituted by at least one group independently selected from halogen (preferably fluoro or chloro), CF₃, or MeO; preferably Y is not substituted or is substituted by at least one halogen group (preferably fluoro or chloro);

Z is —N(OH)CHO.

For example, preferred compounds of the invention include those wherein B is heteroaryl (preferably pyridyl, pyrimidinyl, thienyl, furyl; most preferably pyridyl) and A is phenyl.

Other preferred compounds of the invention include those wherein B is phenyl or heteroaryl (preferably pyridyl, pyrimidinyl, thienyl, furyl; most preferably pyridyl) and A is heteroaryl (preferably pyridyl or pyrimidinyl; most preferably pyrimidinyl).

Other preferred compounds include those wherein R1 is 3- or 4-chlorophenylethyl, 3- or 4-chlorophenylpropyl, 2- or 3-pyridylethyl, 2- or 3-pyridylpropyl, 2- or 4-pyrimidinylethyl (optionally monosubstituted by fluoro or chloro), 2- or 4-pyrimidinylpropyl (optionally monosubstituted by fluoro or chloro), 2-(2-pyrimidinyl)ethyl (optionally monosubstitued by fluoro or chloro), 2-(2-pyrimidinyl)propyl (optionally monosubstitued by fluoro or chloro). Particularly preferred compounds include those wherein R1 is 2-pyrimidinylpropyl, 2-pyrimidinylethyl, and 5-fluoro-2-pyrimidinylethyl.

Particularly preferred compounds of the invention are compounds of the formula II, wherein Z is a reverse hydroxamate group:

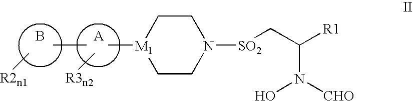

wherein A, B, n1, n2, M₁, R1, R2, R3, X and Y are as defined above for the compound of formula I.

It will be appreciated that the particular substituents and number of substituents on A and/or B and/or R1 are selected so as to avoid sterically undesirable combinations.

Each exemplified compound represents a particular and independent aspect of the invention.

Where optically active centres exist in the compounds of formula I, we disclose all individual optically active forms and combinations of these as individual specific embodiments of the invention, as well as their corresponding racemates.

It will be appreciated that the compounds according to the invention can contain one or more asymmetrically substituted carbon atoms. The presence of one or more of these asymmetric centres (chiral centres) in a compound of formula I can give rise to stereoisomers, and in each case the invention is to be understood to extend to all such stereoisomers, including enantiomers and diastereomers, and mixtures including racemic mixtures thereof.

Where tautomers exist in the compounds of formula I, we disclose all individual tautomeric forms and combinations of these as individual specific embodiments of the invention.

As previously outlined the compounds of the invention are metalloproteinase inhibitors, in particular they are inhibitors of MMP13. Each of the above indications for the compounds of the formula I represents an independent and particular embodiment of the invention. Whilst we do not wish to be bound by theoretical considerations, the compounds of the invention are believed to show selective inhibition for any one of the above indications relative to any MMP1 inhibitory activity, by way of non-limiting example they may show 100-1000 fold selectivity over any MMP1 inhibitory activity.

The compounds of the invention may be provided as pharmaceutically acceptable salts. These include acid addition salts such as hydrochloride, hydrobromide, citrate and maleate salts and salts formed with phosphoric and sulphuric acid. In another aspect suitable salts are base salts such as an alkali metal salt for example sodium or potassium, an alkaline earth metal salt for example calcium or magnesium, or organic amine salt for example triethylamine.

They may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a compound of the formula I or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect the present invention provides a pharmaceutical composition which comprises a compound of the formula I or a pharmaceutically acceptable salt or an in vivo hydrolysable ester and pharmaceutically acceptable carrier.

The pharmaceutical compositions of this invention may be administered in standard manner for the disease condition that it is desired to treat, for example by oral, topical, parenteral, buccal, nasal, vaginal or rectal adminstration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect, the present invention provides a compound of the formula I or a pharmaceutically acceptable salt or in vivo hydrolysable ester thereof for use in a method of therapeutic treatment of the human or animal body. In particular we disclose use in the treatment of a disease or condition mediated by MMP13.

In yet a further aspect the present invention provides a method of treating a metalloproteinase mediated disease condition which comprises administering to a warm-blooded animal a therapeutically effective amount of a compound of the formula I or a pharmaceutically acceptable salt or in vivo hydrolysable ester thereof. Metalloproteinase mediated disease conditions include arthritis (such as osteoarthritis), atherosclerosis, chronic obstructive pulmonary diseases (COPD).

In another aspect the present invention provides processes for preparing a compound of the formula I or a pharmaceutically acceptable salt or in vivo hydrolysable ester thereof which processes are described below.

Where Z is N(OH)CHO, a compound of the formula II is prepared from a compound of the formula III by addition of hydroxylamine followed by formylation. The compound of formula III is prepared conveniently from a compound of the formula IV and a compound of the formula V by cross-coupling methodology where A₁ and B₁ are groups that enable the coupling to occur.

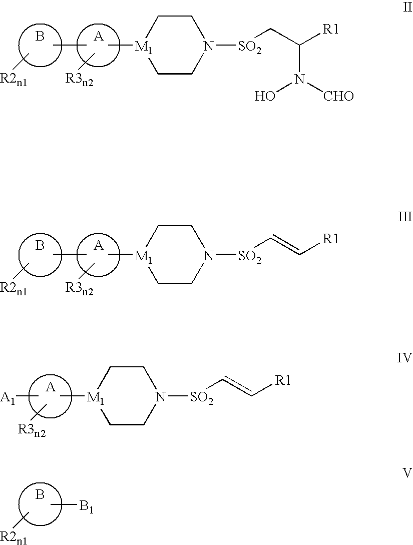

A compound of the formula IV is conveniently prepared by reaction of the sulphonamide of the formula VI with an aldehyde of the formula VIII or with an alkyl or aryl ester of the formula IX. A compound of the formula VI is prepared from a compound of the formula VII.

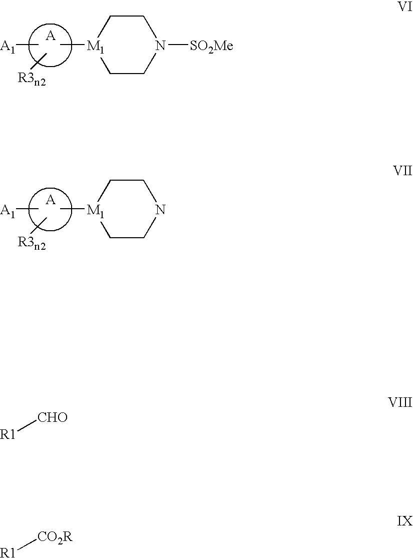

A compound of the formula VII is conveniently prepared from a compound of the formula XI (where P is hydrogen or a suitable protecting group and M₁′ is hydrogen or a suitably reactive group) and a compound of the formula X (where A₂ is a group to enable reaction of X and XI)

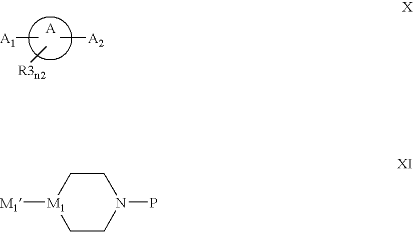

To those skilled in the art it will be clear that ring B could be incorporated into a compound of the formula II at alternative stages of the synthesis.

Where Z is C(O)NHOH, a compound of the formula XII is conveniently prepared from a precursor carboxylic acid (compound of the formula XIII)

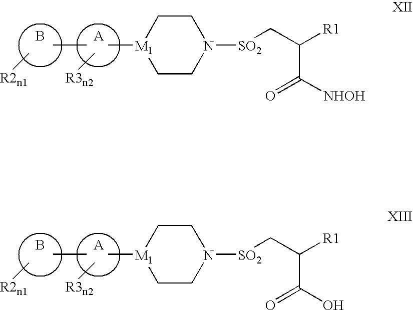

A compound of the formula XIII is prepared from a compound of the formula IV and a compound of the formula XV by cross-coupling methodology where A₁ and B₁ are groups that enable the coupling to occur.

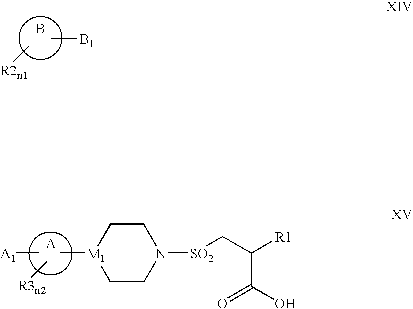

A compound of the formula XV is prepared from compounds of the formulae VI and XVI, where X is a suitable leaving group.

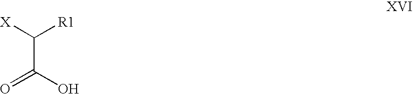

To those skilled in the art it will be clear that ring B could be incorporated into compound XII at alternative stages of the synthesis.

It will be appreciated that many of the relevant starting materials are commercially available or may be made by any convenient method as described in the literature or known to the skilled chemist or described in the Examples herein. In addition the following table shows details of intermediates and their corresponding registry numbers in Chemical Abstracts.

| | Chemical Abstracts |
|---|---|
| | Registry Numbers |
| 4-Pyridylboronic acid | 1692-15-5 |
| 3-Pyridylboronic acid | 1692-25-7 |
| 2-Thiophenboronic acid | 6165-68-0 |
| 3-Thiophenboronic acid | 6165-69-1 |
| 4-Methyl 2-thiophenboronic acid | 162607-15-0 |
| 3-Furanboronic acid | 55552-70-0 |
| 5-Pyrimidine butanal | 260441-11-0 |
| Piperazine, 1-(5-bromo-2-pyridinyl)-4- | 260441-55-2 |
| (methylsulfonyl) | |
| 4-Fluorophenyl boronic acid | 1765-93-1 |
| 4-Chlorophenyl boronic acid | 1679-18-1 |
| 2-(tri-n-butylstannyl)pyridine | 17997-47-6 |
| 2-(tri-n-butylstannyl)thiophene | 54663-78-4 |
| 2-(tri-n-butylstannyl)furan | 118486-94-5 |
| 2-Chlorophenyl boronic acid | 3900-89-8 |
| 4-Ethoxyphenyl boronic acid | 22237-13-4 |
| 4-(Methylthio)phenyl boronic acid | 98546-51-1 |
| 2-(Trifluoromethyl)Phenylboronic Acid | 1423-27-4 |
| 2,4-Difluorophenylboronic Acid | 144025-03-6 |
| 2-Bromophenylboronic Acid | 98437-24-2 |
| 2-Fluorophenyl boronic acid | 1993-03-9 |
| 4-Pyrimidin-2-yl butanal | 260441-10-9 |
| 3-(5-Chloropyrimidin-2-yl)propanal | 357647-90-6 |
| 3-(5-Fluoropyrimidin-2-yl)propanal | 357647-69-9 |
| 3-Pyrimidin-2-yl propanal | 260441-07-4 |
| 3,4-Difluorophenyl boronic acid | 168267-41-2 |
| Pyrimidin-5-yl boronic acid | 109299-78-7 |
| 2,4-Dimethoxy-5-pyrimidinyl boronic | 89641-18-9 |
| acid | |
| 3,5-Difluorophenyl boronic acid | 156545-07-02 |
| 2-Methoxyphenyl boronic acid | 5720-06-9 |
| 4-Trifluoromethylphenyl boronic acid | 128796-39-4 |
| 3-Fluorophenyl boronic acid | 768-35-4 |
| 4-Methoxyphenyl boronic acid | 5720-07-0 |
| 2-Furanboronic acid | 13331-23-2 |
| 3-Trifluoromethyl boronic acid | 1423-26-3 |
| 3-Chlorophenyl boronic acid | 63503-60-6 |
| 3-Cyanophenyl boronic acid | 150255-96-2 |
| 2-Chloro-4-fluorophenylzinc iodide | Rieke Metals, Inc |
| (0.5 M in THF) | |

The compounds of the invention may be evaluated for example in the following assays:

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.2 mM ZnCl and 0.05% (w/v) Brij 35 using the synthetic substrate 7-methoxycoumarin-4-yl)acetyl.Pro.Leu.Gly. Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}-Fourescence_{background}] divided by the [Fluorescence_{minus inhibitor}-Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate

4′,5′-Dimethoxy-fluoresceinyl

Ser.Pro.Leu.Ala.Gln.Ala.Val.Arg. Ser.Ser. Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with 20% piperidne in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity Test as an Agent to Inhibit Membrane Sheddases Such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. Heparinized (10 Units/ml) human blood obtained from volunteers is diluted 1:5 with medium (RPMI1640+bicarbonate, penicillin, streptomycin and glutamine) and incubated (160 μl) with 20 μl of test compound (triplicates), in DMSO or appropriate vehicle, for 30 min at 37° C. in a humidified (5% CO₂/95% air) incubator, prior to addition of 20 μl LPS (E. coli. 0111:B4; final concentration 10 μg/ml). Each assay includes controls of diluted blood incubated with medium alone (6 wells/plate) or a known TNFα inhibitor as standard. The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNFα concentration by ELISA.

Test as an Agent to Inhibit In Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e,g. rats, marmosets) are dosed iv or po with a soluble formulation of compound (such as 20% w/v DMSO, 60% w/v PEG400) and at subsequent time points (e.g. 5, 15, 30, 60, 120, 240, 480, 720, 1220 mins) the blood samples are taken from an appropriate vessel into 10 U heparin. Plasma fractions are obtained following centrifugation and the plasma proteins precipitated with acetonitrile (80% w/v final concentration). After 30 mins at −20° C. the plasma proteins are sedimented by centrifugation and the supernatant fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In Vivo Assessment

Test as an Anti-TNF Agent

The ability of the compounds of this invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα: production by LPS-stimulated human blood. The rat plasma samples are thawed and 175 μl of each sample are added to a set format pattern in a 96 well plate. Fifty μl of heparinized human blood is then added to each well, mixed and the plate is incubated for 30 min at 37° C. (humidified incubator). LPS (25 μl; final concentration 10 μg/ml) is added to the wells and incubation continued for a further 5.5 hours. Control wells are incubated with 25 μl of medium alone. Plates are then centrifuged for 10 min at 2000 rpm and 200 μl of the supernatants are transferred to a 96 well plate and frozen at −20° C. for subsequent analysis of TNF concentration by ELISA.

Data analysis by dedicated software calculates for each compound/dose:

$$Percent inhibition of TNF α = \frac{Mean T N F α ( Controls ) - Mean TNF α ( Treated ) \times 100}{Mean TNF α ( Controls )}$$

Test as an Anti-Arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Test as an Anti-Cancer Agent

Activity of a compound as an anti-cancer agent may be assessed essentially as described in I. J. Fidler (1978) Methods in Cancer Research 15:399-439, using for example the B16 cell line (described in B. Hibner et al., Abstract 283 p75 10th NCI-EORTC Symposium, Amsterdam Jun. 16-19 1998).

The invention will now be illustrated but not limited by the following Examples:

### EXAMPLE 1

Hydroxy[4-pyrimidin-2-yl-1-({[4-(4-thien-3-ylphenyl)piperazin-1-yl]sulfonyl} methyl)butyl]formamide

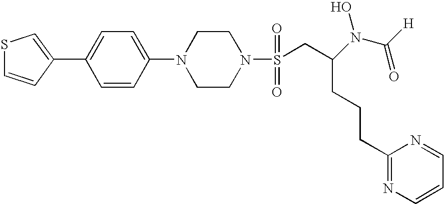

Formic acid (1.44 ml) and acetic anhydride (0.4 ml) were mixed together at 0° C. for 30 minutes, before being added to a solution of 2-(4-(hydroxyamino)-5-{[4-(4-thien-3-ylphenyl)piperazin-1-yl]sulfonyl} pentyl)pyrimidine (105 mg) in tetrahydrofuran (10 ml) and formic acid (0.5 ml) at 0° C. The reaction was allowed to reach room temperature and was stirred overnight, evaporated to dryness and the residue was dissolved in methanol. The solution was stirred overnight and then evaporated to dryness to yield an oil. The oil was triturated with ether to yield a solid, which was collected and dried overnight. Yield 58 mg.

NMR (d6-DMSO@373 k) δ 9.4, br, 1H; 8.7, d, 2H; 8.1, br, 1H; 7.5, m, 2H; 7.4, m, 1H; 7.25, m, 2H; 7.1, m, 1H; 7.0, m, 2H; 3.6-3.3, m,8H; 3.2, m, 1H; 2.9, m, 4H; 1.75, br m, 4H.

MS MH+ 516

The starting material was prepared as follows:

i) To a solution of 1-(4-bromophenyl)piperazine hydrochloride (5.09 g, 18.3 mmol) and triethylamine (7.67 ml) in dichloromethane (100 ml) was added methanesulfonyl chloride (2.83 ml, 36.3 mmol) dropwise. The mixture was stirred for 1 hour at room temperature then dichloromethane (100 ml) was added. The organics were washed with water (2×), brine and dried (Na₂SO₄) and evaporated in vacuo to a yellow solid which crystallised from Ethanol and washed with diethyl ether to give 1-(4-bromophenyl)-4-(methanesulfonyl)piperazine (4.74 g, 81% yield) as a white fluffy powder.

¹H NMR (300 MHz CDCl₃) δ/ppm: 7.38 (d, 2H), 6.91 (d, 2H), 3.21 (m, 8H), 2.89 (s, 3H)

MS: ES+, (M+H)⁺=318, 320 (Br isotope pattern)

ii) To the 1-(4-bromophenyl)-4-(methanesulfonyl)piperazine (902 mg, 2.0 mmol) suspended in anhydrous THF (15 ml), under Nitrogen, cooled to between −20 and −30° C. was added sequentially Lithium bis(trimethylsilyl)amide (1.0M in THF, 4.0 ml), Chlorotrimethylsilane (217 mg, 2.0 mmol, 25311) and 4-pyrimidin-2-ylbutanal (300 mg, 2.0 mmol). The mixture was stirred at −20° C. for 1 hour, quenched with saturated ammonium chloride solution and allowed to stand at ambient temperature overnight. The solvents were removed in vacuo and the residue partitioned between dichloromethane (15 ml) and water (5 ml), the organics separated and chromatogrammed (50 g Silica Bond Elute, eluted with O_{—}100% Ethyl Acetate/Hexane gradient) to give 2-(-5-{[4-(4-bromophenyl)piperazin-1-yl]sulfonyl pent-4-enyl)pyrimidine as a white crystalline material (759 mg, 84% Yield)

MS: ES⁺, (M+H)⁺=451, 453 (Br isotope pattern)

NMR (CDCl₃) δ 8.6, d, 2H; 7.3, m,2H; 7.15, m, 1H; 6.75, m,2H; 6.2, m, 2H; 3.35, m,8H; 3.05, m, 2H; 2.8-2.35, m, 2H; 2.0, m, 2H;

(iii) 2-(−5-{[4-(4-bromophenyl)piperazin-1-yl]sulfonyl}pent-4-enyl)pyrimidine (451 mg) was dissolved in dimethoxy ethane (20 ml) under an argon atmosphere. Thiophene-2-boronic acid (154 mg) and tetrakis(triphenylphosphine)palladium (102 mg) were added, followed by saturated NaHCO3 solution (7 ml). The reaction mixture was refluxed under argon for 3.5 hours, cooled and partitioned between ethyl acetate and water. The organic phase was collected, dried over MgSO4, filtered and evaporated to dryness to yield the crude product 2-(-5-{[4-(4-thien-3-ylphenyl)piperazin-1-yl]sulfonyl}pent-4-enyl)pyrimidine. The crude product was used without further purification, yield 450 mg.

NMR (CDCl3) δ 8.64, d, 1H; 7.7-6.9, m, 9H; 6.1, m, 1H; 3.3, m,8H; 8.05, m, 2H; 2.75-2.4, m, 2H; 2.0, m, 2H.

MS MH+ 455

(iv) The crude alkene 2-(-5-{[4-(4-thien-3-ylphenyl)piperazin-1-yl]sulfonyl}pent-4-enyl)pyrimidine (450 mg) was dissolved in tetrahydrofuran (20 ml) and hydroxylamine (50% in water) (7 ml) was added. The mixture was stirred at ambient temperature overnight. Solvent was removed by evaporation and the residue was partitioned between dichloromethane and water. The organic phase was dried over MgSO4, filtered and evaporated to dryness. The residue was flash column chromatographed, eluting with 2.5% methanol/97.5% ethyl acetate to give 2-(4-(hydroxyamino)-5-{[4-(4-thien-3-ylphenyl)piperazin-1-yl]sulfonyl} pentyl)pyrimidine as a white solid. Yield 200 mg.

NMR d6-DMSO@ 373 K δ 8.65, d, 2H; 7.45, m, 2H; 7.3, m, 1H; 7.25, m, 2H; 7.16, m, 1H; 6.95, m, 3H; 3.4-3.2, m, 10H; 3.05, m, 1H; 2.9, m, 2H; 1.9, m, 2H;1.6, m, 2H.

MS MH+ 488

### EXAMPLE 2

The following analogues were prepared by the method given in Example 1 using the appropriate boronic acid in place of thiophene-2-boronic acid:

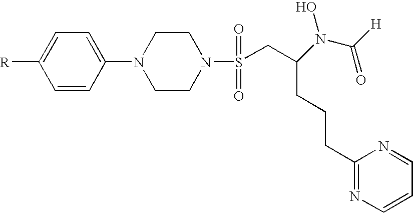

| R | MH+ | NMR d6-DMSO δ |
|---|---|---|
| 4-Pyridyl | 511 | 9.5, br, 1H; 8.7, d, 2H; 8.5, d, 2H; |
| | | 8.15, b, 1H; 7.7, m, 2H; 7.55, m, 2H |
| | | 7.3, m, 1H; 7.1, m, 1H; 3.4, m, 8H; |
| | | 3.2, dd, 1H; 2.9, m, 3H; 1.75, m, 4H. |
| 3-Pyridyl | 511 | 9.4, br, 1H; 8.8, d, 1H; 8.6, d, 1H; |
| | | 8.5, d, 1H; 7.9, m, 1H; 7.55, m, 2H; |
| | | 7.3, m, 1H; 7.2, m, 1H; 7.0, m, 2H |
| | | 3.3, m, 8H, 3.2, m, 1H; 2.85, m, 3H; 1.8, m, 4H. |
| 3-Furan | 500 | 9.75, br, 1H; 8.7, m, 2H; 8.1, m, 2H; |
| | | 7.7, m, 1H; 7.4, m, 2H; 7.2, m, 1H; |
| | | 6.95, m, 2H; 6.85, d, 1H; |
| | | 3.2, m, 10H; 2.9, m, 2H; 1.7, m,4H. |
| 2-Thiophen | 516 | 9.4, br, 1H; 8.7, d, 2H; 8.1, br, 1H; |
| | | 7.5, m, 4H; 7.4, m, 1H; 7.2, m, 1H; |
| | | 6.9, m, 2H; 3.4, m, 4H; 3.25, m, 4H; |
| | | 3.1, m, 1H; 2.9, m, 4H; 1.7, m, 4. |
| 2-(4-methyl)thiophen | 530 | 9.7, br, 1H; 8.7, m, 2H; |
| | | 8.15, br, 1H; 7.5, m, 2H; 7.3, m, 1H; |
| | | 7.2, m, 1H; 6.95, m, 3H; |
| | | 3.3, br m, 10H; 2.9, m, 2H; |
| | | 2.2, s, 3H; 1.7, m, 4H. |

### EXAMPLE 3

1-[({4-[5-(4-fluorophenyl)pyridin-2-yl]piperazin-1-yl} sulfonyl)methyl]-4-pyrimidin-2-ylbutyl(hydroxy)formamide

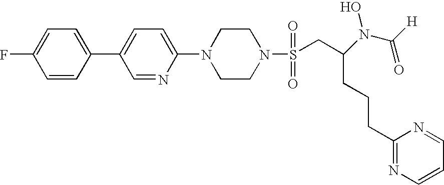

With stirring, under argon, 2-[5-({4-[5-(4-fluorophenyl)pyridin-2-yl]piperazin-1-yl}sulfonyl)-4-(hydroxyamino)pentyl]pyrimidine (365 mg, 0.73 mmol) was dissolved in tetrahydrofuran (3.5 ml)/formic acid (1.75 ml). With ice cooling was added dropwise a preformed mixture of formic acid (880 μl) and acetic anhydride (410 μl, 4.38 mmol). The mixture was allowed to stir at room temperature for 1 hour before the solvents were evaporated and the residue dissolved in dichloromethane and washed with saturated sodium hydrogen carbonate solution. The organic layer was dried (Mg2SO4), evaporated and treated with methanol (10 ml) at 50° C. for 30 minutes, then evaporated and chromatogrammed by semi-prep HPLC (8 μm Hyperprep HS C18 (250 mm×21.2 mm), eluent H₂O/MeCN/MeOH/TFA 67.5/12.5/20/0.5) to give the title compound as a white powder (97 mg, 25% yield)

NMR (400 Mz, DMSO-d6, 373K), δ/ppm: 9.40 (1H, br s), 8.68 (2H, m), 8.42 (1H, d), 8.13 (1H, br s), 7.83 (1H, m), 7.62 (2H, m), 7.23 (3H, m), 6.93 (1H, d), 4.80-4.10 (1H, br s), 3.68 (4H, m), 3.46 (1H, dd), 3.30 (4H, m), 3.18 (1H, dd), 2.91 (2H,t), 1.90-1.65 (4H, m)

Mass: ES+ (M+H)+ 529

The starting material was prepared as follows:

(i) 2-(5-{[4-(5-bromopyridin-2-yl)piperazin-1-yl]sulfonyl} pent-4-enyl)pyrimidine

Prepared as a mixture of E and Z geometrical isomers using the method given in example 1(ii)-using 1-(5-bromo-pyridin-2-yl)-4-(methanesulfonyl)piperazine in place of 1-(4-bromophenyl)-4-(methanesulfonyl)piperazine

NMR (300 Mz, DMSO-d6, 273K), δ/ppm: 8.71(2H, m), 8.19 (1H, m), 7.71 (1H, m), 7.33 (1H, m), 6.87 (1H, m), 6.65 (*), 6.47 (1H, m), 6.30 (1,d), 3.60 (4H, m), 3.09 (4H, m), 2.88 (2H, dd), 2.57 (1H, dd), 2.29 (1H,t), 1.91 (2H, m) * minor geometrical isomer

(ii) 2-[(5-({4-[5-(4-fluorophenyl)pyridin-2-yl]piperazin-1-yl} sulfonyl)pent-4-enyl]pyrimidine

Under argon, a flask was charged with 4-fluorophenyl boronic acid (232 mg, 1.66 mmol), Bis(triphenylphosphine)palladium chloride (15.4 mg, 0.022 mmol) and 2-(5-{[4-(5-bromopyridin-2-yl)piperazin-1-yl]sulfonyl}pent-4-enyl)pyrimidine(500 mg, 110 mmol). To this were added toluene (10 ml) and potassium carbonate (401 mg, 2.9 mmol) in water (5 ml) and the mixture stirred at 75° C., under argon, for 4 days. The mixture was cooled and added to water (50 ml), then extracted with dichloromethane (2×50 ml). The extracts were combined, dried, evaporated and chromatogrammed on silica (50 g, EtOAc eluent) to give the title compound as a white powder (406 mg, 79%)

Mass: ES+ (M+H)+=468

(iii) 2-[5-({4-[5-(4-fluorophenyl)pyridin-2-yl]piperazin-1-yl} sulfonyl)-4-(hydroxyamino)pentyl]pyrimidine

Under argon, hydroxylamine (50% solution in water, 460 μl) was added to a stirred solution of 2-[(5-({4-[5-(4-fluorophenyl)pyridin-2-yl]piperazin-1-yl} sulfonyl)pent-4-enyl]pyrimidine (350 g, 0.75 mmol) in THF (6 ml) and the mixture stirred at room temperature overnight. The solvent was evaporated and the residue azeotroped with toluene (2×20 ml) and triturated with diethylether to give the title compound as a white powder (375 mg, 100%)

Mass: ES+ (M+H)+=501

### EXAMPLE 4

1-[(4-[5-(4-chlorophenyl)pyridin-2-yl]piperazin-1-yl} sulfonyl)methyl]-4-pyrimidin-2-ylbutyl(hydroxy)formamide

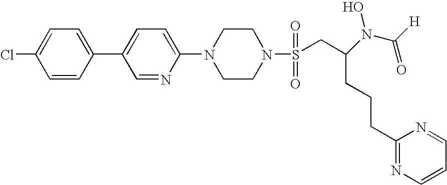

By analogy to Example 3 the title compound was prepared.

NMR (400 Mz, DMSO-d6, 373K), δ/ppm: 9.45 (1H, br s), 8.70 (2H, d), 8.46 (1H, d), 8.15 (1H, br s), 8.89 (1H, dd), 7.62 (2H, dd), 7.48 (2H, dd), 7.29 (1H, t), 6.96 (2H, d), 4.80-4.05 (1H, br s), 3.66 (4H, t), 3.45 (1H, dd), 3.31 (4H, t), 2.88 (2H,t), 1.90-1.60 (4H, m)

Mass: ES+ (M+H)+=545, 547 (Cl isotope pattern)

### EXAMPLE 5

1-[({4-[5-(3-furyl)pyridin-2-yl]piperazin-1-yl} sulfonyl)methyl]-4-pyrimidin-2-ylbutyl(hydroxy)formamide

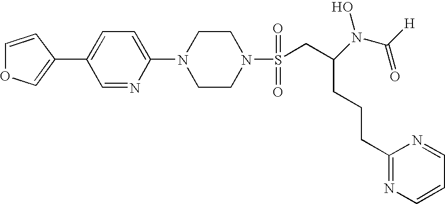

By analogy to Example 3 the title compound was prepared.

NMR (400 Mz, DMSO-d6, 373K), δ/ppm: 9.45 (1H, br s), 8.70 (2H, d), 8.42 (1H, d), 8.14 (1H, br s), 8.01 (1H, s), 7.77 (2H, m), 7.68 (1H, s), 7.29 (1H, t), 6.95 (2H, m) 4.90-3.95 (1H, br s), 3.65 (4H, t), 3.44 (1H, dd), 3.32 (4H, t), 3.18 (1H, dd), 2.89 (2H,t), 1.90-1.60 (4H, m)

Mass: ES+ (M+H)+=501

### EXAMPLE 6

1-({[4-(2,3′-bipyridin-6′-yl)piperazin-1-yl]sulfonyl}methyl)-4-pyrimidin-2-ylbutyl(hydroxy)formamide

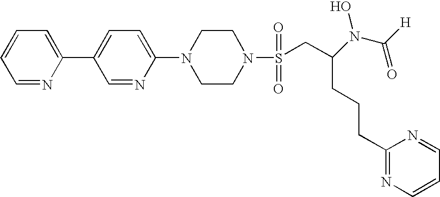

Under Argon, a preformed mixture of formic acid (1.0 ml) and acetic anhydride (378μl, 408 mg, 4.0 mmol) was added dropwise to a solution of 6′-(4-{[2-(hydroxyamino)-5-pyrimidin-2-ylpentyl]sulfonyl} piperazin-1-yl)-2,3′-bipyridine (103 mg, 0.21 mmol) in THF (5 ml)/formic acid (2.5 ml), cooled to 0° C. The mixture was allowed to warm to room temperature and stirred for 1 hour. The solvents were then evaporated and the residue dissolved in dichloromethane (20 ml) and stirred with saturated sodium bicarbonate solution (10 ml) for 1 hour. The organics were separated and purified on silica (20 g, EtOAc eluent) to give the title compound as a white powder (60 mg, 56% yield)

NMR (300 Mz, DMSO-d6, 373K), δ/ppm: 9.45 (1H, br s), 8.85 (1H, s), 8.70 (2H, d), 8.63 (1H, d), 8.25-7.98 (2H, m), 7.82 (2H, m), 7.28 (2H, m), 6.98 (1H, d), 4.80-4.00 (1H, br s), 3.72 (4H, t), 3.42 (1H, dd), 3.33 (4H, t), 3.19 (1H, dd), 2.89 (2H,t), 1.90-1.70 (4H, m)

Mass: ES+ (M+H)+=512

The starting material was prepared as follows:

6′-(4-{[2-(hydroxyamino)-5-pyrimidin-2-ylpentyl]sulfonyl} piperazin-1-yl)-2,3′-bipyridine

Under argon, hydroxylamine (50% solution in water, 0.5 ml) was added to a solution of 6′-(4-{[5-pyrimidin-2-ylpent-1-enyl]sulfonyl} piperazin-1-yl)-2,3′-bipyridine (96 mg, 0.21 mmol) in dry THF (4.0 ml) and the mixture stirred at room temperature overnight. Evaporation of the solvents yielded the title compound as a yellow powder (103 mg, 100% yield)

Mass: ES+ (M+H)+=484

6′-(4-{[5-pyrimidin-2-ylpent-1-enyl]sulfonyl} piperazin-1-yl)-2,3′-bipyridine

Under argon, 2-(5-{[4-(5-bromopyridin-2-yl)piperazin-1-yl]sulfonyl} pent-4-enyl)pyrimidine (226 mg, 0.5 mmol) and tetrakis(triphenylphosphine)palladium (29 mg, 0.025 mmol) were dissolved in dry toluene (10 ml) and to the stirred solution was added 2-(tri-n-butylstannyl)pyridine (276 mg, 0.75 mmol) in dry toluene (1 ml). The mixture was heated to 95° C. overnight, cooled and then was added potassium fluoride (2M, 2.0 ml) and the mixture stirred at room temperature for 5 hours. The mixture was extracted with dichloromethane (10 m]) and the organic layer passed through a PTFE robot filter, evaporated and chromatogrammed on silica gel (2.5% Methanol/Dichloromethane eluent) to give a pale yellow powder (100 mg, 44%)

Mass: ES+ (M+H)+=451

### EXAMPLE 7

hydroxy[4-pyrimidin-2-yl-1-({[4-(5-thien-2-ylpyridin-2-yl)piperazin-1-yl]sulfonyl} methyl)butyl]formamide

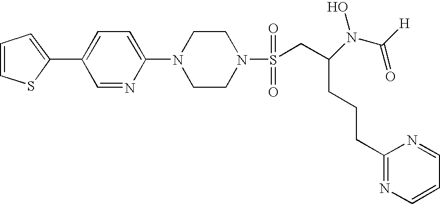

By analogy with Example 5, the title compound was obtained as a white powder (80 mg, 35%)

NMR (300 Mz, DMSO-d6, 373K), δ/ppm: 9.40 (1H, br s), 8.69 (2H, d), 8.44 (1H, d), 8.25-7.98 (1H, m), 7.80 (1H, dd), 7.43 (1H, dd), 7.33 (1H, dd), 7.29 (1H, t), 7.10 (1H, t), 6.90 (1H, d), 4.80-4.00 (1H, br s), 3.67 (4H, t), 3.44 (1H, dd), 3.32 (4H, t), 3.18 (1H, dd), 2.89 (2H,t), 1.87-1.63 (4H, m)

### EXAMPLE 8

1-[({4-[5-(2-furyl)pyridin-2-yl]piperazin-1-yl} sulfonyl)methyl]-4-pyrimidin-2-ylbutyl(hydroxy)formamide

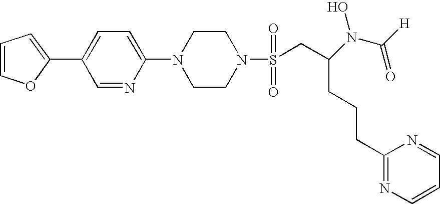

By analogy with example 5, the title compound was obtained as a white powder (56 mg, 27%)

Mass: ES+ (M+H)+=501

NMR (500 Mz, DMSO-d6, 373K), δ/ppm: 9.39 (1H, br s), 8.67 (2H, d), 8.47 (1H, d), 8.10 (1H, br s), 7.80 (1H, dd), 7.60 (1H, d), 7.24 (1H, t), 6.89 (1H, d), 6.68 (1H, d), 6.51 (1H, dd), 4.40 (1H, br s), 3.65 (4H, t), 3.43 (1H, dd), 3.29 (4H, t), 3.17 (1H, dd), 2.88 (2H,t), 1.85-1.63 (4H, m)

### EXAMPLE 9

1-[({4-[5-(4-fluorophenyl)pyrazin-2-yl]piperazin-1-yl} sulfonyl)methyl]-4-pyrimidin-2-ylbutyl(hydroxy)formamide

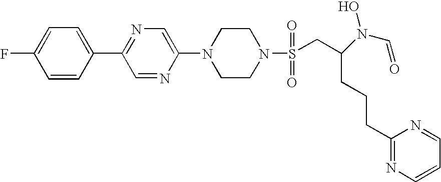

Formic acid (1.40 ml) and acetic anhydride (0.38 ml) were mixed together at 0° C. for 30 minutes, before being added to a solution of 2-[5-({4-[5-(4-fluorophenyl)pyrazin-2-yl]piperazin-1-yl} sulphonyl)-4-(hydroxyamino)pentyl}pyrimidine (290 mg) in tetrahydrofuran (10 ml) and formic acid (1.0 ml) at 0° C. The reaction was allowed to reach room temperature and was stirred overnight, neutralised with saturated sodium bicarbonate solution and extracted with dichloromethane. The organic phase was dried over magnesium sulphate, filtered, evaporated to dryness and the residue was dissolved in methanol. The solution was stirred overnight and then evaporated to dryness to yield an oil. The oil was triturated with ether to yield, 1-[({4-[5-(4-fluorophenyl)pyrazin-1-yl]piperazin-1-yl} sulphonyl)methyl]-4-pyrimidin-2-lbutyl(hydroxy)formamide. Yield 210 mg.

NMR (DPX400 CD3Cl) δ9.7, br d,1H; 8.7,m,3H; 8.4,d,1H; 8.0,m,2H; 7.3,m,3H; 4.7-4.2,d,1H; 3.8,m,4H; 3.3,br m,6H; 2.9,m.2H; 1.7,br m,4H.

MS MH+ 530.03

The starting material was prepared as follows

i) To a solution of 2-chloro-5-(4-fluorophenyl)pyrazine (3.45 g) {CA Reg No 115104-61-5} in dimethylacetamide (25 ml) was added anhydrous piperazine (4.4 g). The solution was stirred at 120° C. overnight. Cooled and evaporated in vacuo to an oily solid. Stirred in ethyl acetate for 1 hour. The insoluble material was removed by filtration. The organic filtrate was dried over magnesium sulphate, filtered and evaporated to yield 2-(4-fluorophenyl)-5-piperazin-1-ylpyrazine. Yield 4.1 g

NMR (DPX400 CD3Cl) δ 8.5, d,1H; 8.2,d,1H; 7.8,m,2H; 7.1,d,2H; 3.65,m,4H; 3.1,m,4H

MS MH+ 259.06

ii) To a solution of 2-(4-fluorophenyl)-5-piperazin-1-ylpyrazine (2.58 g, 0.01M) and triethylamine (4.2 ml) in dichloromethane (100 ml) at 0° C. was added methanesulphonyl chloride (0.96 ml, 0.0.011 M) dropwise. The mixture was stirred overnight at room temperature, then dichloromethane (100 ml) was added. The organics were washed with water, dried (Magnesium sulphate) and evaporated in vacuo to a yellow solid which crystallised from ethanol to give 2-(4-fluorophenyl)-5-[4-(methylsulphonyl)piperazin-1-yl]pyrazine. Yield 2.7 g.

NMR (400 MHz CD3Cl) δ 8.5,d,1H; 8.2, d,1H; 7.9,m,2H; 7.15,m,2H;,3.8,m,4H;3.4,m,4H; 2.85,s,3H.

MS MH+ 337.01

iii) To the 2-(4-fluorophenyl)-5-[4-(methylsulphonyl)piperazin-1-yl]pyrazine (840 mg,0.0025M) dissolved in anhydrous THF (200 ml), under argon, and cooled to −10° C. was added Lithium bis(trimethylsilyl)amide (1.0M in THF 5.5 ml 0.0055M). Diethyl chorophosphate (0.37 ml, 00025M) and a solution of 4-pyrimidin-2-ylbutanal (375 mg, 0.0025M) in dry THF(5 ml) were added sequentially. The mixture was stirred at −10° C. for 1 hour, quenched with saturated ammonium chloride solution and extracted with ethyl acetate. The organic phase was dried over magnesium sulphate, filtered and evaporated to an oily solid. Chromatographed on Merck 9385 silica, eluting with ethyl acetate to yield 2-[4-5-({4-[5-(4-fluorophenyl)pyrazin-1-yl}pent-4-enyl]pyrimidine as a solid. Yield 325 mg.

NMR 400 MHz CD3Cl δ 8.7,m,2H; 8.5, s,1H; 7.9,m,2H; 7.15, m,2H; 6.85,m,1H; 6.4,m, 6.1,dd,2H; 3.8,m,4H; 3.3,m,H;3.1,m,2H; 2.75-2.3 dm,2H;2.5,m,2H.

MS MH+ 469.03

iv) The alkene 2-[4-5-({4-[5-(4-fluorophenyl)pyrazin-1-yl}pent-4-enyl]pyrimidine (310 mg) was dissolved in tetrahydrofuran (10 ml) and hydroxylamine (50% in water) (2 ml) was added. The mixture was stirred at ambient temperature overnight. The reaction mixture was partitioned between saturated ammonium chloride solution and dichloromethane. The organic phase was dried over magnesium sulphate, filtered and evaporated to give 2-[5-({4-[5-(4-fluorophenyl)pyrazin-1-yl} sulphonyl)-4-(hydroxyamino]pyrimidine as a white solid. Yield 297 mg

NMR 400 MHz CD3Cl δ 8.65, d, 2H; 8.5, d, 1H; 8.15, d, H; 7.8,m, 2H; 7.2, m, 2H; 3.73, m, 4H; 3.4, m, 5H; 3.2-2.9, m, 2H; 1.9, m,2H; 1.65,m,2H.

MS MH+ 502.03

### EXAMPLE 10

hydroxy[1-({[4-(5-phenylpyrazin-2-yl)piperazin-1-yl]sulfonyl} methyl)-4-pyrimidin-2-ylbutyl]formamide

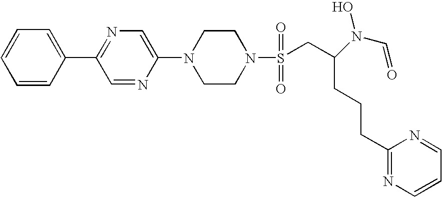

By analogy with example 9, the above compound was synthesised starting from the analogous chloropyrazine CA Reg No 25844-73-9

MS MH+ 512.05

### EXAMPLE 11

1-[({4-[5-(3-furyl)pyridin-2-yl]piperazin-1-yl} sulfonyl)methyl]-4-pyrimidin-2-ylbutyl(hydroxy)formamide

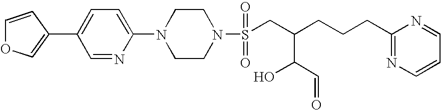

To a ice-cooled solution of 2-[5-({4-[5-(3-furyl)pyridin-2-yl]piperazin-1-yl} sulfonyl)-4-(hydroxyamino)pentyl]pyrimidine (426 mg, 0.90 mmol) in a mixed sovent system of THF/formic acid (6 ml/2 ml) was added a preformed mixture of formic acid (2.0 ml) and acetic anhydride (1 ml). The mixture was then stirred at room temperature for 1 hour. The solvents were evaporated and the residue partitioned between dichloromethane (15 ml) and saturated Sodium Bicarbonate solution (10 ml) and stirred at ambient temperature overnight. The organic layer was then separated using a PTFE (0.45 micron) robot filter, evaporated and the residue was purified by flash chromatography (silica gel, 10 g, 0-10% EtOH/EtOAc) to give the title compound as a white powder (266 mg, 59% yield)

NMR (400 Mz, DMSO-D6, 373K), δ/ppm: 9.39 (1H, br s), 8.68 (2H, d), 8.40 (1H, d), 8.13 (1H, br s), 7.99 (1H, t), 7.76 (1H, dd), 7.67 (1H, t), 7.27 (1H, t), 6.85 (2H, dd), 4.40 (1H, br s), 3.64 (4H, t), 3.44 (1H, dd), 3.32 (4H, t), 3.17 (1H, t), 2.91 (2H, t), 1.77 (4H, m)

Mass: ES+ (M+H)+=501

Chiral chromatography: The enantiomers of 1-[({4-[5-(3-furyl)pyridin-2-yl]piperazin-1-yl}sulfonyl)methyl]-4-pyrimidin-2-ylbutyl(hydroxy)formamide were resolved on Daicel Chiralpak AD 2 cm×25 cm column with eluent of 10% MeOH/MeCN

The starting material was prepared as follows:

i) 2-[(4E. Z)-5-({4-[5-(3-furyl)pyridin-2-yl]piperazin-1-yl} sulfonyl)]ent-4-enyl]pyrimidine

To a stirred solution of 2-((4E,Z)-5-{[4-(5-bromopyridin-2-yl)piperazin-1-yl]sulfonyl}pent-4-enyl)pyrimidine (440 mg, 0.97 mmol) in DME (20 ml), under Argon at RT, was added 3-furylboronic acid (134 mg, 1.2 mmol), tetrakis(triphenylphoshine)palladium (102 mg, 10 mol %) and saturated sodium bicarbonate solution (7 ml). The mixture was heated to reflux for 3 hours. After cooling to room temperature, the mixture was partitioned between dichloromethane (20 ml) and water (10 ml). The organic phase was separated using a PTFE (0.45 micron) robot filter and purified by flash chromatography (silica gel, 20 g, 50-100% EtOAc/iso-hexane) to give the title compound as a pale yellow solid (407 mg, 95%).

NMR (400 Mz, DMSO-D6, 373K), δ/ppm: 8.67 (2H,d), 8.46 (1H, d), 7.88 (1H, dd), 7.64 (1H, m), 7.47 (2H, d), 7.31 (1H, t), 6.96 (1H, d), 6.69 (1H, m), 6.50 (1H, d), 3.67 (4H, t), 3.11 (4H, t), 2.87 (2H, t), 2.30 (2H, m), 1.93 (2H, m)

Mass: ES+ (M+H)+=440

ii) 2-[5-({4-[5-(3-furyl)pyridin-2-yl]piperazin-1-yl}sulfonyl)-4-(hydroxyamino)pentyl]pyrimidine

A stirred solution of 2-[(4E,Z)-5-({4-[5-(3-furyl)pyridin-2-yl]piperazin-1-yl}sulfonyl)pent-4-enyl]pyrimidine (395 mg, 0.90 mmol) in THF (10 ml), under Argon, was treated at room temperature with hydroxylamine (50% solution in H₂O, 1.0 ml) for 2.5 hours. The solvents were evaporated to give the title compound, 426 mg, 99% Mass: ES+ (M+H)⁺=473

### EXAMPLE 12

The following compounds were prepared using the method given in Example 11.

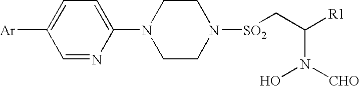

| Ar | R1 | M + H |
|---|---|---|
| 3-Pyridyl | 2-PyrimidinylCH2CH2CH2 | 512.5 |
| 4-Pyridyl | 2-PyrimidinylCH2CH2CH2 | 512.5 |
| 3,4-difluorophenyl | 2-PyrimidinylCH2CH2CH2 | 547.5 |
| Thien-3-yl | 2-PyrimidinylCH2CH2CH2 | 517.5 |
| 4-fluorophenyl | 2-PyrimidinylCH2CH2CH2 | 529.4 i. |
| 4-fluorophenyl | 5-F-2-PyrimidinylCH2CH2 | 533.3 |
| Pyrimidin-5-yl | 2-PyrimidinylCH2CH2CH2 | 513.1 |
| 2,4-difluorophenyl | 2-PyrimidinylCH2CH2CH2 | 547.0 |
| 2-chlorophenyl | 2-PyrimidinylCH2CH2CH2 | 545.0 & |
| | | 547.0 ii. |
| 2-fluorophenyl | 2-PyrimidinylCH2CH2CH2 | 529.0 |
| 2,4-di-MeO-pyrimidin-5-yl | 2-PyrimidinylCH2CH2CH2 | 573.1 |

NOTES
i. Resolved enantiomers using Daicel Chiralpak AD 2cm × 25 cm 10%MeOH/MeCN eluent
ii. Chlorine isotope pattern

### EXAMPLE 13

1-[({4-[5-(4-fluorophenyl)pyrimidin-2-yl]piperazin-1-yl} sulfonyl)methyl]-3-pyrimidin-2-ylpropyl(hydroxy)formamide

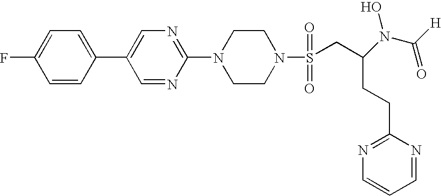

Formic acid (2.63 mL, 70 mmol) and acetic anhydride (0.7 mL, 7 mmol) were mixed together at 0° C. for 30 minutes, before being added to a solution of 5-(4-fluorophenyl)-2-(4-{[2-(hydroxyamino)-4-pyrimidin-2-ylbutyl]sulfonyl}piperazin-1-yl)pyrimidine (690 mg, 1.4 mmol) in tetrahydrofuran (10 mL) and formic acid (2.63 mL) at 0° C. The reaction was allowed to reach room temperature and was stirred for 45 minutes. The reaction was then evaporated in vacuo, and azeotroped with toluene (2×5 mL). The residue was dissolved in MeOH and heated to 45° C. for one hour. The solution was then evaporated in vacuo, and the residue triturated with Et₂O to give a white solid which was collected by filtration, washed with Et₂O and dried in vacuo to give 1-[({4-[5-(4-fluorophenyl)pyrimidin-2-yl]piperazin-1-yl} sulfonyl)methyl]-4-pyrimidin-2-ylbutyl(hydroxy)formamide as a white solid (417 mg, 57%).

¹H NMR (d6-DMSO@373 k) δ 9.45 (br, s, 1H), 8.68 (m, 4H), 8.09 (br, s, 1H), 7.67 (m, 2H), 7.28 (m, 3H), 4.41 (br, s, 1H), 3.91 (m, 4H), 3.49 (dd, 1H), 3.33 (m, 4H), 3.29 (dd, 1H), 2.87 (m, 2H), 2.21 (m, 2H).

MS (ESI): 516.43 (MH⁺)

The starting material was prepared as follows:

To a stirred solution of tert-butyl 4-(5-bromopyrimidin-2-yl)piperazine-1-carboxylate (15.5 g, 45.5 mmol, CAS number 374930-88-8) and 4-fluorophenyl-boronic acid (7.63 g, 54.5 mmol) in a mixed solvent system of DME:saturated aqueous sodium bicarbonate solution (200 mL: 160 mL) at RT was added Pd(PPh₃)₄ (2.6 g, 2.25 mmol). The reaction was then stirred for 3 hours at 90° C., before being cooled to RT. The reaction was then quenched with water (200 mL) and the layers were separated. The aqueous phase was extractd with EtOAc (3×200 mL) and the combined organic extracts were dried (MgSO₄), filtered and evaporated in vacuo. The residue was then purified by flash chromatography (silica gel, 50% EtOAc in hexanes) to give tert-butyl 4-[5-(4-fluorophenyl)pyrimidin-2-yl]piperazine-1-carboxylate as a silvery solid (16.6 g, 45 mmol, 98%).

¹H NMR (CDCl₃) δ: 8.50 (s, 2H), 7.43 (m, 2H), 7.12 (m, 2H), 3.86 (m, 4H), 3.52 (m, 4H), 1.52 (s, 9H).

MS (ESI): 303.30 (MH⁺-t-Bu)

To a stirred solution of tert-butyl 4-[5-(4-fluorophenyl)pyrimidin-2-yl]piperazine 1-carboxylate (16.5 g, 46.1 mmol) in CH₂Cl₂ (50 mL) at RT was added trifluoroacetic acid (40 mL). The mixture was then stirred vigorously at RT for 1 hour. Volatiles were removed in vacuo, and the residue was azeotroped with toluene (2×50 mL). The crude residue was then dissolved in CH₂Cl₂ (150 mL) and cooled to 0° C. Triethylamine (19.2 mL, 0.13 mol) was then added, followed by dropwise addition of methanesulfonyl chloride (3.9 mL, 50 ml). The reaction was then allowed to stir at RT for one hour, before being quenched by the addition of water (100 mL). The layers were separated, and the aqueous phase extracted with CH₂Cl₂ (2×100 mL). The combined organic extracts were dried (MgSO₄), filtered and evaporated in vacuo to give 5-(4-fluorophenyl)-2-[4-(methylsulfonyl)piperazin-1-yl]pyrimidine as a colourless solid (12.84 g, 83%).

¹H NMR (CDCl₃) δ: 8.52 (s, 2H), 7.44 (m, 2H), 7.16 (m, 2H), 4.07 (m, 4H), 3.34 (m, 4H), 2.82 (s, 3H).

MS (ESI): 337.02 (MH⁺)

To a stirred suspension of 5-(4-fluorophenyl)-2-[4-(methylsulfonyl)piperazin-1-yl]pyrimidine (504 mg, 1.5 mmol) in THF (15 mL) at −78° C., was added dropwise a solution of LiHMDS in THF (3.1 mL, 1.0M solution, 3.1 mmol). The resulting suspension was stirred at −78° C. for 30 minutes before being treated with diethyl chlorophosphate (0.23 mL, 1.6 mmol). The solution was then maintained at −78° C. for 30 minutes before being warmed slowly to −20° C. The reaction was then treated with a solution of 4-pyrimidin-2-ylbutanal (220 mg, 1.6 mmol) in THF (2 mL). The solution was then maintained at −20° C. for one hour before being quenched with saturated aqueous ammonium chloride solution (5 mL) The layers were separated and the aqueous phase extracted with ethyl acetate (3×5 mL). The combined organic extracts were then dried, (MgSO₄), filtered and concentrated in vacuo to give 5-(4-fluorophenyl)-2-(4-{[(1E/Z)-4-pyrimidin-2-ylbut-1-enyl]sulfonyl}piperazin-1-yl)pyrimidine as a brown solid which was used crude in the next step.

MS (ESI): 455.40 (MH⁺)

To a stirred solution of 5-(4-fluorophenyl)-2-(4-{[(1E/Z)-4-pyrimidin-2-ylbut-1-enyl]sulfonyl}piperazin-1-yl)pyrimidine (crude from previous step) in THF (10 mL) at RT was added 50% aqueous hydroxylamine (1.5 mL) and the mixture stirred rapidly for 2 hours. The reaction was quenched by the addition of saturated ammonium chloride solution (5 mL) and the layers were then separated. The aqueous phase was extracted with EtOAc (3×5 mL) and the combined organic extracts were then dried (MgSO₄), filtered and evaporated in vacuo. The white solid obtained was then purified by flash chromatography (silica gel, 5% MeOH in CH₂Cl₂), to give 5-(4-fluorophenyl)-2-(4-{[2-(hydroxyamino)-4-pyrimidin-2-ylbutyl]sulfonyl} piperazin-1-yl)pyrimidine as a white solid (698 mg, 1.48 mmol, 95% over two steps).

¹H NMR (d6-DMSO) δ: 8.72 (m, 4H), 7.67 (m, 2H), 7.29 (m, 3H), 5.68 (br s, 1H), 4.01 (m, 4H), 3.89 (m, 4H), 3.40 (dd, 1H), 3.31 (m, 5H), 3.11 (m, 2H), 2.11 (m, 2H).

MS (ESI): 488.42 (MH⁺).

### EXAMPLE 14

(1S)-1-[({4-[5-(4-fluorophenyl)pyrimidin-2-yl]piperazin-1-yl} sulfonyl)methyl]-3-pyrimidin-2-ylpropyl(hydroxy)formamide

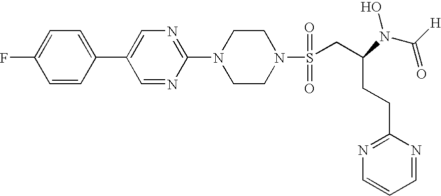

The racemic mixture, prepared as in example 13, was separated by chiral HPLC (on a Chiralcel OJ column, 10 m, 2 cm×25 cm, flow rate 9 ml/min eluent=EtOH) to give (1S)-1-[({4-[5-(4-fluorophenyl)pyrimidin-2-yl]piperazin-1-yl} sulfonyl)methyl]-4-pyrimidin-2-ylbutyl(hydroxy)formamide as a white solid

¹H NMR (d6-DMSO@373 k) δ 9.45 (br, s, 1H), 8.68 (m, 4H), 8.09 (br, s, 1H), 7.67 (m, 2H), 7.28 (m, 3H), 4.41 (br, s, 1H), 3.91 (m, 4H), 3.49 (dd, 1H), 3.33 (m, 4H), 3.29 (dd, 1H), 2.87 (m, 2H), 2.21 (m, 2H).

MS (ESI): 516.43 (MH⁺)

### EXAMPLE 15

The following compounds were also prepared using the method given in example 13.

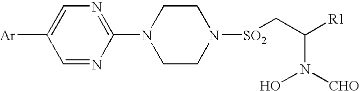

| Ar | R1 | M + H |
|---|---|---|
| 4-F-Ph | 5-Cl-2-PyrimidinylCH2CH2 | 550.38 |

### EXAMPLE 16

(1S)-1-[({4-[5-(4-fluorophenyl)pyrimidin-2-yl]piperazin-1-yl} sulfonyl)methyl]4-pyrimidin-2-ylbutyl(hydroxy)formamide

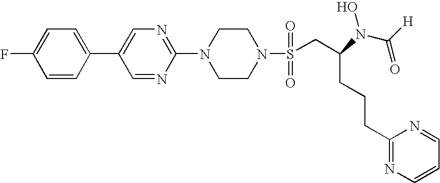

Formic acid (0.37 mL, 10 mmol) and acetic anhydride (0.2 mL, 2 mmol) were mixed together at 0° C. for 30 minutes, before being added to a solution of 5-(4-fluorophenyl)-2-(4-{[2-(hydroxyamino)-5-pyrimidin-2-ylpentyl]sulfonyl}piperazin-1-yl)pyrimidine (240 mg, 0.48 mmol) in tetrahydrofuran (3 mL) and formic acid (0.37 mL) at 0° C. The reaction was allowed to reach room temperature and was stirred for 45 minutes. The reaction was then evaporated in vacuo, and azeotroped with toluene (2×5 mL). The residue was then dissolved in MeOH and heated to 45° C. for one hour. The solution was then evaporated in vacuo, and the residue triturated with Et₂O to give a white solid which was collected by filtration, washed with Et₂O and dried in vacuo (182 mg, 70%). The racemic mixture was then separated by chiral HPLC (on a Merck Chiralpak AS-V column, 20 μm, 5 cm×25 cm, flow rate 35 ml/min eluent=90% EtOH/10% MeCN/MeOH) to give (1S)-1-[({4-[5-(4-fluorophenyl)pyrimidin-2-yl]piperazin-1-yl} sulfonyl)methyl]4-pyrimidin-2-ylbutyl(hydroxy)formamide as a white solid.

¹H NMR (d6-DMSO@373 k) δ 9.4 (br, s, 1H), 8.62 (m, 4H), 8.11 (br, s, 1H), 7.66 (m, 2H), 7.21 (m, 3H), 4.55 (br, s, 1H), 3.88 (m, 4H), 3.45 (dd, 1H), 3.30 (m, 4H), 3.16 (m, 1H), 2.89 (m, 2H), 1.68 (m, 4H).

MS (ESI): 530.28 (MH⁺)

The starting material was prepared as follows

To a stirred solution of 5-bromo-2-piperazin-1-ylpyrimidine (22.38 g, 92 mmol, CAS number 99931-82-5) and triethylamine (38.5 mL, 276 mmol) in dichloromethane (400 mL) at 0° C. was added methanesulfonyl chloride (10.7 mL, 138 mmol) dropwise over 10 minutes. The reaction was then stirred for 30 minutes at 0° C., before being allowed to warm to RT and stirred for an additional 30 minutes. The reaction was then quenched with water (200 mL) and the layers were separated. The organic phase was washed with water (200 mL) and the organics were dried (MgSO₄), filtered and evaporated in vacuo. The residue was then triturated with ethyl acetate and the solid residue filtered and dried in vacuo to give 5-bromo-2-[4-(methylsulfonyl)piperazin-1-yl]pyrimidine as an off white solid (22.4 g, 69.6 mmol, 76%).

¹H NMR (CDCl₃) δ: 8.30 (s, 2H), 3.96 (m, 4H), 3.28, (m, 4H), 7.67 (dd, 1H), 2.81 (s, 3H).

MS (ESI): 321.18 (MH⁺)

To a stirred suspension of 5-bromo-2-[4-(methylsulfonyl)piperazin-1-yl]pyrimidine (21.36 g, 66.5 mmol) in THF (700 mL) at −78° C., was added dropwise a solution of LiHMDS in THF (146 mL, 1.0M solution, 0.146 mol). The resulting suspension was stirred at −78° C. for 30 minutes before being treated with diethyl chlorophosphate (10.6 mL, 73.2 mmol). The solution was then maintained at −78° C. for 30 minutes before being warmed slowly to −20° C. The reaction was then treated with a solution of 4-pyrimidin-2-ylbutanal (11 g, 73.2 mmol) in THF (50 mL). The solution was then maintained at −20° C. for one hour before being quenched with saturated aqueous ammonium chloride solution (500 mL). The layers were separated and the aqueous phase extracted with ethyl acetate (3×300 mL). The combined organic extracts were then dried, (MgSO₄), filtered and concentrated in vacuo to give a brown soild which was purified by flash chromatography (silica gel, 25% to 50% to 100% EtOAc in hexanes) to give 5-bromo-2-(4-{[(1E/Z)-5-pyrimidin-2-ylpent-1-enyl]sulfonyl}piperazin-1-yl)pyrimidine as a yellow solid (13 g, 43%, E:Z 1.89:1).

¹H NMR (CDCl₃) δ: 8.68 (m, 2H), 8.27 (m, 2H), 7.13, (m,1H), 6.82 (ddd, 1H), 6.35 (ddd)*, 6.11 (ddd, 1H), 5.95 (ddd)*, 3.90 (m, 4H), 3.17 (m, 4H), 3.09 (m, 2H), 2.72 (m)*, 2.34 (m, 2H), 2.11 (m, 2H) * minor geometrical isomer.

MS (ESI): 454.95 (MH⁺ Br isotope pattern).

A stirred solution of 5-bromo-2-(4-{[(1E/Z)-5-pyrimidin-2-ylpent-1-enyl]sulfonyl}piperazin-1-yl)pyrimidine, (453 mg, 1 mmol), Pd(PPh₃)₄ (115 mg, 0.1 mmol) and 4-fluorophenyl boronic acid (166 mg, 1.2 mmol) in a mixed solvent system of DME/saturated aqueous sodium hydrogencarbonate (10 mL:7 mL) was heated to 95° C. for 3 hours. The mixture was then cooled to room temperature and partitioned between water and EtOAc (5 mL:5 mL). The layers were separated, and the aqueous phase extracted with EtOAc (3×5 mL). The combined organic extracts were then dried ((MgSO₄), filtered and evaporated in vacuo. The solid residue was used crude in the next step.

MS (ESI): 469.00 (MH⁺)

To a stirred solution of 5-(4-fluorophenyl)-2-(4-{[(1E/Z)-5-pyrimidin-2-ylpent-1-enyl]sulfonyl}piperazin-1-yl)pyrimidine (crude from previous step) in THF (10 mL) at RT was added 50% aqueous hydroxylamine (2 mL) and the mixture stirred rapidly for 2 hours. The reaction was quenched by the addition of saturated ammonium chloride solution (5 mL) and the layers were then separated. The aqueous phase was extracted with EtOAc (3×5 mL) and the combined organic extracts were then dried (MgSO₄), filtered and evaporated in vacuo. The white solid obtained was then purified by flash chromatography (silica gel, 50% to 100% EtOAc in hexanes), to give 5-(4-fluorophenyl)-2-(4-{[2-(hydroxyamino)-5-pyrimidin-2-ylpentyl]sulfonyl}piperazin-1-yl)pyrimidine as a white solid (245 mg, 0.488 mmol, 49% over two steps).

¹H NMR (CDCl₃) δ: 8.66 (m, 2H), 8.50 (s, 2H), 7.42 (m, 2H), 7.11 (m, 3H), 5.44 (br s, 1H), 4.01 (m, 4H), 3.44 (m, 5H), 3.21 (m, 1H), 2.94 (m, 1H), 2.82 (dd, 1H), 2.07 (m, 1H), 1.94 (m, 1H), 1.77 (m, 1H), 1.60 (m, 1H).

MS (ESI): 502.02 (MH⁺)

### EXAMPLE 17

1-[({4-[5-(2-chloro-4-fluorophenyl)pyrimid-2-yl]piperazin-1-yl} sulfonyl)methyl]-4-pyrimidin-2-ylbutyl(hydroxy)formamide

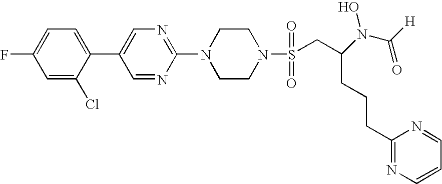

With stirring, under argon, 2-[5-({4-[5-(2-chloro-4-fluorophenyl)pyrimid-2-yl]piperazin-1-yl} sulfonyl)-4-(hydroxyamino)pentyl]pyrimidine (260 mg, 0.485 mmol) was dissolved in dichloromethane (2.5 ml)/formic acid (1 ml). With ice cooling was added dropwise a mixture of formic acid (1 ml) and acetic anhydride (200 μl) preformed at 8° C. The mixture was allowed to stir at room temperature for 20 minutes before the solvents were evaporated and azeotroped with toluene. The residue was dissolved in dichloromethane (5 ml) and treated with methanol (5 ml) at room temperature for 18 hours. The solution was evaporated, diluted with dichloromethane and azeotroped several times with diethyl ether to give the title compound as a white powder (248 mg, 91% yield)

NMR (300 MHz, DMS0-d6, 373K), δ/ppm: 8.65 (2H, d), 8.45 (2H, s), 7.5 (2H, m), 7.3 (2H, m), 3.9 (4H, b s), 3.45 (1H, m), 3.30 (4H, b s), 3.15 (1H, dd), 2.9 (2H, b), 1.75 (4H, b)

Mass: ES+ (M+H)+ 564, 566 (Cl isotope pattern)

The starting material was prepared as follows:

(i) 2-[(5-({4-[5-(2-chloro-4-fluorophenyl)pyrimid-2-yl]piperazin-1-yl} sulfonyl)pent-4-enyl]pyrimidine

To stirred 2-(5-{[4-(5-bromopyrimid-2-yl)piperazin-1-yl]sulfonyl}pent-4-enyl)pyrimidine (453 mg, 1 mmol) were added in three aliquots at reaction times of 0, 1 and 5 hrs, Tetrakis(triphenylphospine)palladium (3×46 mg, total 120 μmol) and 2-Chloro-4-fluorophenyl zinc iodide (2×1.1 ml & 1.5 ml, 0.5M in THF, 1.85 mmol). After the initial additions, the reaction was heated at 50° C. The mixture was quenched with water (2 ml), sodium hydrogen carbonate (sat., 2 ml) added and diluted with ethyl acetate. The suspension was filtered and washed well with ethyl acetate. The filtrate was washed with water and brine, back-extracting with ethyl acetate. Dried (MgSO4) and filtered through silica (2 g) washing well with ethyl acetate to give the title compound a mixture of E/Z geometrical isomers as a brown oil (558 mg, 93% @ 84 wt %)

NMR (300 MHz, CDCl3), δ/ppm: 8.65 (2H, t), 8.4 (2H, s), 7.3 (obscured by PPh3O), 7.15 (1H, t), 7.05 (2H, m), 6.85, (0.4H, dt), 6.5, (0.6H, dt), 6.15, (0.4H, d), 6.05 (0.6H, d), 4.0 (4H, t), 3.25 (4H, t), 3.0 (2H, q), 2.75 (1.2H, q), 2.35 (0.8H, q), 2.05 (2H, obs)

Mass: ES+ (M+H)+=503, 505 (Cl isotope pattern)

(ii) 2-[5-({4-[5-(2-chloro-4-fluorophenyl)pyrimid-2-yl]piperazin-1-yl} sulfonyl)-4-(hydroxyamino)pentyl]pyrimidine

Under argon, hydroxylamine (50% solution in water, 567 μl) was added to a stirred solution of 2-[(5-({4-[5-(2-chloro-4-fluorophenyl)pyrimid-2-yl]piperazin-1-yl}sulfonyl)pent-4-enyl]pyrimidine (554 mg, 0.925 mmol) in tetrahydrofuran (4.5 ml) and the mixture stirred at room temperature overnight. The solution was partitioned between ethyl acetate (2×) and brine. The organic phases were dried (MgSO4) and evaporated, triturated with diethyl ether and decanted. The solid white residue was redissolved in dichloromethane, evaporated to a low volume and triurated with diethyl ether to give the title compound as a white powder (264 mg, 53%)

NMR (300 MHz, CDCl3), δ/ppm: 8.65 (2H, d), 8.4 (2H, s), 7.25 (2H & CHCl3), 7.15 (1H, t), 7.1 (1H, td), 5.5 (1H, b s), 4.0 (4H, t), 3.5 (1H), 3.45 (1H, d), 3.35 (4H, t), 3.2 (1H, p), 3.05 (1H, p), 2.85 (1H, p), 2.05 (1H, m), 1.95 (1H, m), 1.7 (1H, m), 1.6 (1H, m)

Mass: ES+ (M+H)+=536, 538 (Cl isotope pattern)

### EXAMPLE 18

The following analogues were prepared by an analogous manner to that given in Example 16, using the appropriate boronic acid and aldehyde in place of 4-fluorophenyl boronic acid and 4-pyrimidin-2-ylbutanal:

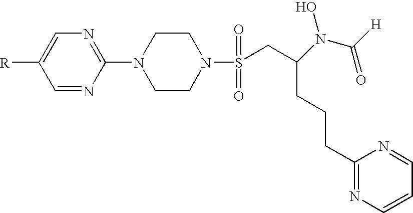

| R | MH+ |
|---|---|
| 2-Chlorophenyl | 546/548 |
| 2-Methoxyphenyl | 542 |
| 4-Ethoxyphenyl | 584 |
| 4-(Methylthio)phenyl | 558 |
| 2-(Trifluoromethyl)Phenyl | 580 |
| 2,4-Difluorophenyl | 548 |
| 4-(Trifluoromethyl)Phenyl | 580 |
| 4-Chlorophenyl | 546.4 |
| 3,4-Difluorophenyl | 548.41 |
| 2-thienyl | 518.43 |
| 2-Bromophenyl | 590/592 |

| Structure | MH+ |
|---|---|
| 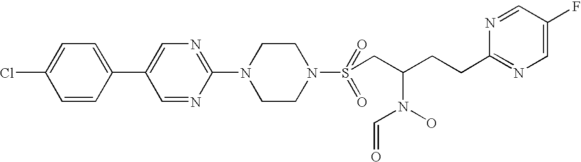 | 550.26 |
| 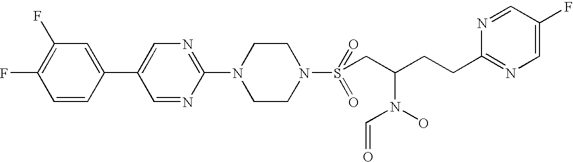 | 552.35 |
| 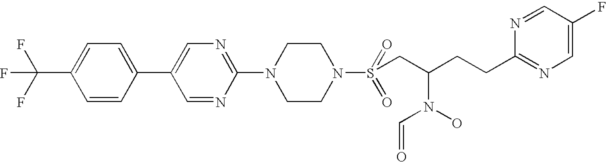 | 584.45 |
| 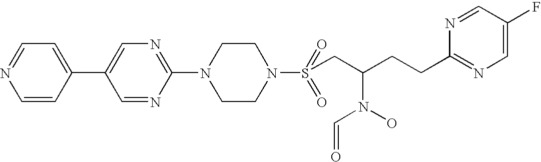 | 517.42 |
| 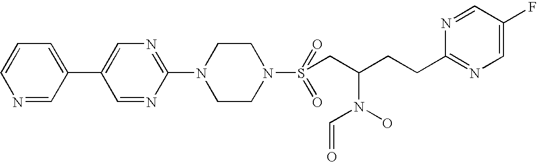 | 517.4 |
| 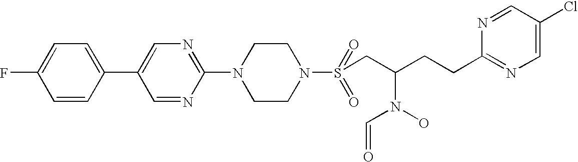 | 550.38 |
| 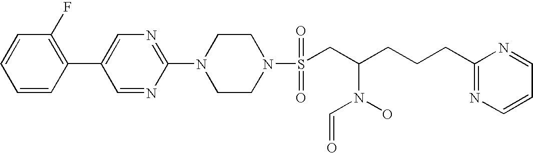 | 530.4 |
| 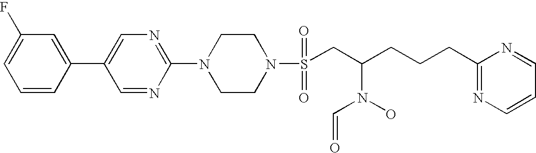 | 530.4* |
| 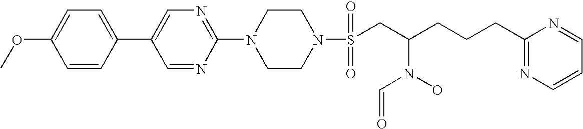 | 542.41 |
| 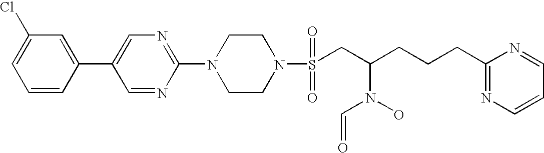 | 546.37 |
| 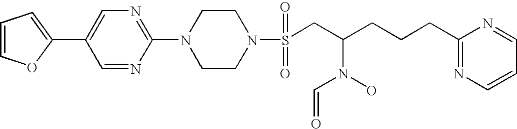 | 502.41 |
| 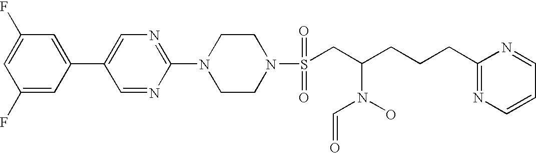 | 548.4 |
| 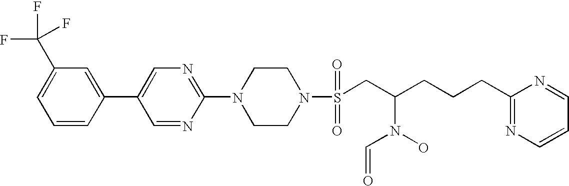 | 580.43 |
| 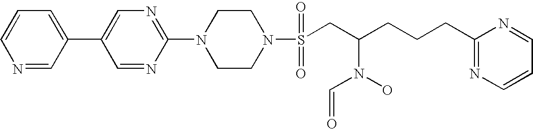 | 513.41** |
| 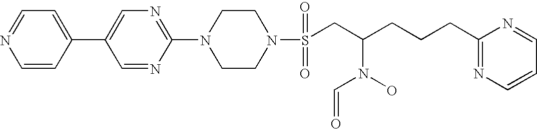 | 513.42** |
| 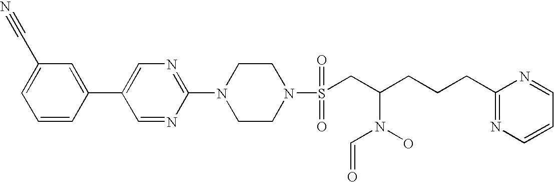 | 537.34 |
| 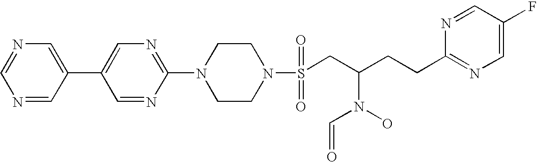 | 518.28 |
| 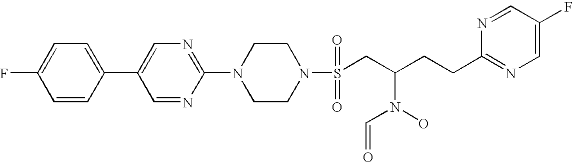 | 534.4 |
| 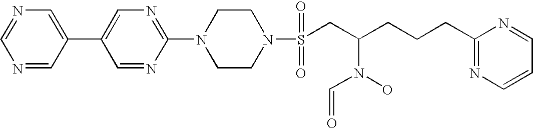 | 514.08 |

The compounds marked were resolved on a chiral column, using the conditions shown below
*Column: Merck 50 mm 20 μm Chiralpak ASV No.ASV00SC-JG001 / Eluant: MeOH
**Column: 20 μm Merck 50 mm Chiralpak AS No.ASV00SC-JG001 / Eluant: MeOH
***Column: 20 μm Merck 50mm Chiralpak AD No.AD00SC-HL001 / Eluant: MeOH/MeCN 50/50

### EXAMPLE 19

(1R or 1S)-1-[({4-[5-(2,4-difluorophenyl)pyrimid-2-yl]piperazin-1-yl}sulfonyl)methyl]-4-pyrimidin-2-ylbutyl(hydroxy)formamide

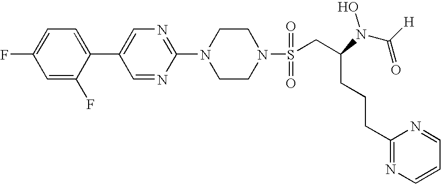

Racemate (250 mg, see example 18) was chromatogrammed (preparative Chiral-AS [Chiral Technologies Europe] HPLC column, eluted with 5% acetonitrile in methanol. Yield 71 mg.

ES+ (M+H)+ 548)

### EXAMPLE 20

The following compounds were prepared in an analogous manner to that given in Example 16 using the appropriate boronic acid in place of 4-fluorophenyl boronic acid:

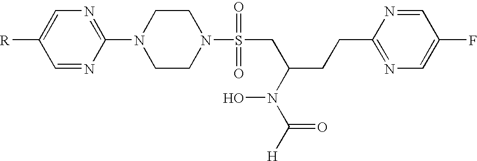

| R | MH+ |
|---|---|
| 2-Chlorophenyl | 550/552 |
| 2-Fluorophenyl | 534 |

### EXAMPLE 21

1-[({4-[5-(2-Fluorophenyl) pyrimidin-2-yl]piperazin-1-yl} sulfonyl)methyl]-3-pyrimidin-2-ylpropyl(hydroxy)formamide.

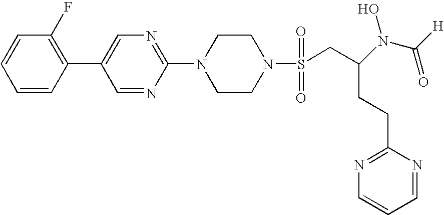

Formic acid (3.2 ml) and acetic anhydride (0.8 ml) were mixed together at 0° C. for 30 minutes, before being added to a crude solution of 5-(2-fluorophenyl)-2-(4-{[2-(hydroxyamino)-4-pyrimidin-2-ylbutyl]sulfonyl}piperazin-1-yl)pyrimidine (740 mg) in tetrahydrofuran (15 ml) at 0° C. The reaction was allowed to reach room temperature and was stirred overnight, evaporated to dryness and the residue was dissolved in methanol. The solution was stirred at 40° C. for 3 hours and then evaporated to dryness to yield an oil. The oil was triturated with diethyl ether to yield 1-[({4-[5-(2-fluorophenyl) pyrimidin-2-yl]piperazin-1-yl} sulfonyl)methyl]-3-pyrimidin-2-ylpropyl(hydroxy)formamide as a white solid. Yield 580 mg. 82% yield over 3 steps.

NMR (d6-DMSO@278 k) δ 9.95 & 9.6, m, 1H; 8.65, s, 2H; 8.3 & 7.9, d, 1H; 7.7-7.5, m, 4H; 7.4, m, 1H; 7.25, m, 3H; 4.2 & 4.8, m, 1H; 3.8-4.0, m, 4H; 3.6-3.4, m, 1H; 3.3, m, 4H; 2.9, m, 2H; 2.1, br m, 2H.

MS MH+ 516

The starting material was prepared as follows:

ii) To 5-bromo-2-[4-(methylsulfonyl)piperazin-1-yl]pyrimidine [see example 16] (8.2 g, 25.5 mmol) suspended in anhydrous tetrahydrofuran (250 ml), under nitrogen, cooled to between −60 and −65° C. was added sequentially lithium bis(trimethylsilyl)amide (1.0M in, tetrahydrofuran 51.0 ml, 51 mmol), with stirring for 20 minutes at −60° C., followed by diethyl chlorophosphonate (3.7 ml, 25.5 mmol), with stirring for 20 minutes and then allowed to warm to −20° C. before addition of a solution 3-pyrimidin-2-ylpropanal (3.2 g, 23.0 mmol) in anhydrous tetrahydrofuran (20 ml). The mixture was stirred at −20° C. for 1 hour, quenched with saturated ammonium chloride solution and allowed to warm to ambient temperature. The reaction mixture was diluted with water (100 ml) and ethyl acetate (100 ml), transferred to a separating funnel the aqueous wash separated and back extracted with ethyl acetate (2×100 ml). The combined organic extracts washed with saturated brine (150 ml), dried over magnesium sulphate. The ethyl acetate was removed in vacuo to give 5-bromo-2-(4-{[(1E)-4-pyrimidin-2-ylbut-1-enyl]sulfonyl}piperazin-1-yl)pyrimidine as a white crystalline material isolated by triturating with ethanol (5.6 g, 50% Yield).

MS: ES⁺, (M+H)⁺=440, 442 (Br isotope pattern)

NMR (d6-DMSO@278 k) δ 8.7-8.6, m, 2H; 8.5, m, 2H; 7.4-7.2, m, 1H; 6.8-6.2, m, 2H; 3.8, m, 4H; 3.1, m, 4H; 2.9, m, 2H; 2.7, m, 2H;

(iii) 5-Bromo-2-(4-{[(1E/Z)-4-pyrimidin-2-ylbut-1-enyl]sulfonyl}piperazin-1-yl)pyrimidine (600 mg) was dissolved in dimethoxymethane (40 ml) under an argon atmosphere. 2-fluorophenyl-boronic acid (154 mg) and tetrakis(triphenylphosphine)palladium (132 mg) were added, followed by saturated sodium hydrogen carbonate solution (20 ml). The reaction mixture was refluxed under argon for 2.5 hours, cooled and partitioned between ethyl acetate and water. The organic phase was collected, dried over magnesium sulphate, filtered and evaporated to dryness to yield the crude product 5-(2-fluorophenyl)-2-(4-{[(1E/Z)-4-pyrimidin-2-ylbut-1-enyl]sulfonyl}piperazin-1-yl) pyrimidine. The crude product (˜750 mg) was used without further purification.

NMR (d6-DMSO@278 k) δ 8.6, m 2H; 7.5, m, 3H; 7.4-7.15, m, 4H; 6.8-6.4, m, 2H; 3.8, m, 4H; 3.05, m, 2H; 2.9, m, 4H; 2.7, m, 2H.

MS MH+ 455

(iv) The crude 5-(2-fluorophenyl)-2-(4-{[(1 E/Z)-4-pyrimidin-2-ylbut-1-enyl]sulfonyl}piperazin-1-yl)pyrimidine (˜750 mg) was dissolved in tetrahydrofuran (15 ml) and hydroxylamine (50% in water) (10 ml) was added. The mixture was stirred at ambient temperature overnight. Solvent was removed by evaporation and the residue was partitioned between ethyl acetate (50 ml) and water (20 ml), the aqueous wash back extracted with ethyl acetate (2×50 ml). The organic phases combined, washed with brine (75 ml) and dried over magnesium sulphate, filtered and evaporated to dryness to give crude 5-(2-fluorophenyl)-2-(4-{[2-(hydroxyamino)-4-pyrimidin-2-ylbutyl]sulfonyl}piperazin-1-yl)pyrimidine. Yield 740 mg.

NMR (d6-DMSO@278 k) δ 8.6, m, 2H; 8.7, m, 2H; 7.7-7.5, m, 4H; 7.4, m, 1H; 7.25, m, 3H; 5.8, m, 1H; 3.8-4.0, m, 4H; 3.4, m, 1H; 3.3-2.9, m, 6H; 2.1-1.9, br m, 2H.

MS MH+ 488

### EXAMPLE 22

The following compounds were prepared by the method given in Example 21 using the appropriate boronic acid in place of 2-fluorophenylboronic acid:

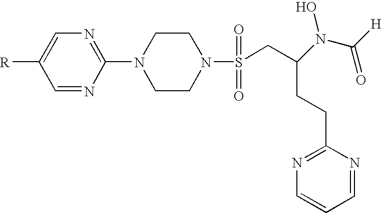

| R | MH+ |
|---|---|
| 2-Chlorophenyl | 532 |
| 2,4-Difluorophenyl | 534 |
| 3,5-Difluorophenyl | 534 |
| 3-Pyridyl | 499 |
| 4-Pyridyl | 499 |

### EXAMPLE 23

hydroxy[1-({[4-(5-pyridin-2-ylpyrimidin-2-yl)piperazin-1-yl]sulfonyl}methyl)-4-pyrimidin-2-ylbutyl] formamide

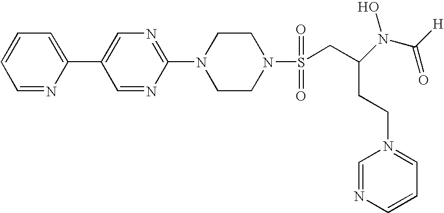

Formic acid (1.8 mL, 50 mmol) and acetic anhydride (0.45 mL, 5 mmol) were mixed together at 0° C. for 30 minutes, before being added to a solution of 2-(4-{[2-(hydroxyamino)-5-pyrimidin-2-ylpentyl]sulfonyl} piperazin-1-yl)-5-pyridin-2-ylpyrimidine (crude from previous step) in tetrahydrofuran (5 mL) at 0° C. The reaction was allowed to reach room temperature and was stirred for 45 minutes. The reaction was then evaporated in vacuo, and azeotroped with toluene (2×5 mL). The residue was dissolved in MeOH and heated to 45° C. for one hour. The solution was then evaporated in vacuo, and the residue was purified by flash chromatography (silica gel, 1% to 5% MeOH in CH₂Cl₂) to give hydroxy[1-({[4-(5-pyridin-2-ylpyrimidin-2-yl)piperazin-1-yl]sulfonyl} methyl)-4-pyrimidin-2-ylbutyl] formamide as a white solid (214 mg, 0.41 mmol 43% over 3 steps).

NMR (d6-DMSO@373 k) δ 9.40 (br, s, 1H), 9.05 (s, 2H), 8.68 (m, 3H), 8.14 (br, s, 1H), 7.85 (m, 2H), 7.29 (m, 2H), 4.40 (vbr, s, 1H), 3.95 (m, 4H), 3.47 (dd, 1H), 3.33 (m, 4H), 3.19 (dd, 1H), 2.90 (m, 2H), 1.76 (m, 4H).

MS (ESI): 513.51 (MH⁺)

The starting material was prepared as follows:

To a stirred solution of tert-butyl 4-(5-bromopyrimidin-2-yl)piperazine-1-carboxylate (4.9 g, 14.3 mmol, CAS number 374930-88-8) 2-(tributylstannyl)pyridine (7.9 g, 21.45 mmol, CAS number 17997-47-6) in DMF (50 ml) was added tetraethylammonium chloride (2.36 g, 14.3 mmol), potassium carbonate (1.98 g, 14.3 mmol) and bis(triphenylphosphine)palladium(II) chloride (0.5 g, 0.71 mmol). The reaction was then stirred under an atmosphere of argon for 2 hours at 100° C. before being cooled to RT. The reaction was filtered through a 0.45 um nylon filter and diluted with water (100 ml), extracted the aqueous with EtOAc (2×50 ml) and the combined organic extracts were dried (MgSO₄), filtered and evaporated in vacuo. The residue was then purified by flash chromatography (90 g Biotage silica gel cartridge, 10% to 40% EtOAc in hexanes) to give tert-butyl 4-(5-pyridin-2-ylpyrimidin-2-yl)piperazine-1-carboxylate as a white solid (1.40 g, 4.1 mmol, 28%).

NMR (CDCl₃) δ 8.95 (s, 2H), 8.64 (d, 1H), 7.73 (m, 1H), 7.59 (d, 1H), 7.20 (m, 1H), 3.90 (m, 4H), 3.52 (m, 4H), 1.49 (s, 9H).

MS (ESI): 286.02 (MH⁺-t-Bu)

To a stirred solution of tert-butyl 4-(5-pyridin-2-ylpyrimidin-2-yl)piperazine-1-carboxylate (1.39 g, 4.1 mmol) in CH₂Cl₂ (20 mL) at RT was added trifluoroacetic acid (4 mL). The mixture was then stirred vigorously at RT for 1 hour. Volatiles were removed in vacuo, and the residue was azeotroped with toluene (3×10 mL). The crude residue was then dissolved in CH₂Cl₂ (20 mL) and cooled to 0° C. Triethylamine (1.7 mL, 12.3 mmol) was then added, followed by dropwise addition of methanesulfonyl chloride (0.35 mL, 4.5 mmol). The reaction was then allowed to stir at RT for one hour, before being quenched by the addition of water (10 mL). The layers were separated, and the aqueous phase extracted with CH₂Cl₂ (2×10 mL). The combined organic extracts were dried (MgSO₄), filtered and evaporated in vacuo to give a yellow gum which was stirred with ethanol and filtered to give 2-[4-(methylsulfonyl)piperazin-1-yl]-5-pyridin-2-ylpyrimidine as a white solid (0.61 g, 47%).

¹H NMR (d6-DMSO) δ: 9.18 (s, 2H), 8.63 (d, 1H), 7.93 (d, 1H), 7.87 (m, 1H), 7.31 (m, 1H), 3.93 (m, 4H), 3.20 (m, 4H), 2.89 (s, 3H).

MS (ESI): 320.33 (MH⁺)

To a stirred suspension of 2-[4-(methylsulfonyl)piperazin-1-yl]-5-pyridin-2-ylpyrimidine (300 mg, 0.94 mmol) in THF (10 mL) at −10° C., was added dropwise a solution of LiHMDS in THF (1.9 mL, 1.0M solution, 1.9 mmol). The resulting suspension was stirred at −10° C. for 30 minutes before being treated with diethyl chlorophosphate (0.135 mL, 0.94 mmol). The solution was then maintained at −10° C. and then treated with a solution of 4-pyrimidin-2-ylbutanal (155 mg, 1.04 mmol) in THF (1 mL). The solution was then maintained at −10° C. for 30 minutes before being quenched with saturated aqueous ammonium chloride solution (5 mL). The layers were separated and the aqueous phase extracted with ethyl acetate (2×5 mL). The combined organic extracts were then dried, (MgSO₄), filtered and concentrated in vacuo to give 5-pyridin-2-yl-2-(4-{[(1E/Z)-5-pyrimidin-2-ylpent 1-enyl]sulfonyl}piperazin-1-yl)pyrimidine as a cream solid which was used crude in the next step.

MS (ESI): 452.0 (MH⁺)

To a stirred solution of 5-pyridin-2-yl-2-(4-{[(1E/Z)-5-pyrimidin-2-ylpent-1-enyl]sulfonyl} piperazin-1-yl)pyrimidine (crude from previous step) in THF (5 mL) at RT was added 50% aqueous hydroxylamine (1.0 mL) and the mixture stirred rapidly for 2 hours. The reaction was quenched by the addition of saturated ammonium chloride solution (5 mL) and the layers were then separated. The aqueous phase was extracted with EtOAc (2×5 mL) and the combined organic extracts were then dried (MgSO₄), filtered and evaporated in vacuo to give 2-(4-{[2-(hydroxyamino)-5-pyrimidin-2-ylpentyl]sulfonyl}piperazin-1-yl)-5-pyridin-2-ylpyrimidine as a white solid which was used crude in the next step.

MS (ESI): 485.49 (MH⁺)

### EXAMPLE 24

3-(5-fluoropyrimidin-2-yl)-1-({[4-(5-pyridin-2-ylpyrimidin-2-yl)piperazin-1-yl]sulfonyl}methyl)propyl(hydroxy)formamide

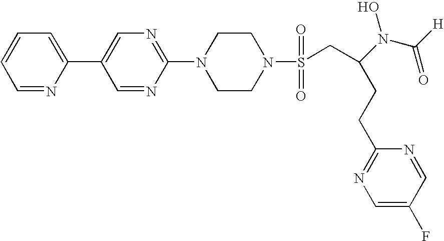

The title compound was prepared using an analogous method to that given in example 23—replacing 4-pyrimidin-2-ylbutanal by 3-(5-fluoro-pyrimdin-2-yl)propanal.

MH+ 517.44

What we claim is:

1. A compound of the formula I or a pharmaceutically acceptable salt or an in vivo hydrolysable ester thereof, 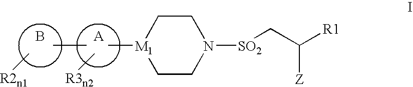
wherein
A and B are each independently selected from phenyl and up to C6 heteroaryl;
wherein
at least one of A and B is heteroaryl;
n1 and n2 are each independently selected from 0, 1, 2, and 3;
each R2 and each R3 is independently selected from OH, NO₂, CF₃, CN, halogen, SC₁₋₄alkyl, SOC₁₋₄alkyl, SO₂C₁₋₄alkyl, C₁₋₄alkyl, and C₁₋₄alkoxy;
M₁ is selected from N and C;
R1 is the group —X—Y;
X is C₁₋₆alkyl;
Y is selected from up to C10 cycloalkyl, up to C10 aryl, and up to C10 heteroaryl; wherein
Y is optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, and C₁₋₄alkoxy; and
Z is selected from —N(OH)CHO, and —C(O)NHOH.

2. A compound of the formula II or a pharmaceutically acceptable salt or an in vivo hydrolysable ester thereof, 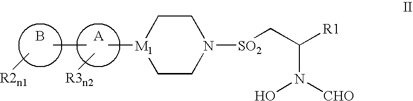
wherein
A and B are each independently selected from phenyl and up to C6 heteroaryl; wherein
at least one of A and B is heteroaryl;
n1 and n2 are each independently selected from 0, 1, 2, 3;
each R2 and each R3 is independently selected from OH, NO₂, CF₃, CN, and halogen,
SC₁₋₄alkyl, SOC₁₋₄alkyl, SO₂C₁₋₄alkyl, C₁₋₄alkyl, and C₁₋₄alkoxy;
M₁ is selected from N and C;
R1 is the group —X—Y;
X is C₁₋₆alkyl; and
Y is selected from up to C₁₀ cycloalkyl, up to C₁₀ aryl, and up to C₁₀ heteroaryl; wherein
Y is optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, and C₁₋₄alkoxy.

3. A compound as claimed in claim 1 or claim 2 or a pharmaceutically acceptable salt or an in vivo hydrolysable ester thereof, wherein at least one of A and B is a five- or six-membered aromatic ring containing one or more heteroatoms independently selected from N, O, and S.

4. A compound as claimed in claim 3 or a pharmaceutically acceptable salt or an in vivo hydrolysable ester thereof, wherein at least one of A and B is pyridyl, pyrimidinyl, thienyl, or furyl.

5. A compound as claimed in claim 1 or claim 2 or a pharmaceutically acceptable salt or an in vivo hydrolysable ester thereof, wherein B is not substituted or B is substituted by at least one R2 group selected from CF₃, CN, halogen, and C₁₋₄alkyl.

6. A compound as claimed in claim 1 or claim 2 or a pharmaceutically acceptable salt or an in vivo hydrolysable ester thereof, wherein A is not substituted or A is substituted by at least one R3 group selected from CF₃, CN, halogen, and C₁₋₄alkyl.

7. A compound as claimed in claim 1 or claim 2 or a pharmaceutically acceptable salt or an in vivo hydrolysable ester thereof, wherein M₁ is N.

8. A compound as claimed in claim 1 or claim 2 or a pharmaceutically acceptable salt or an in vivo hydrolysable ester thereof, wherein X is C₂₋₅alkyl.

9. A compound as claimed in claim 8 or a pharmaceutically acceptable salt or an in vivo hydrolysable ester thereof, wherein X is C₂₋₃alkyl.

10. A compound as claimed in claim 1 or claim 2 or a pharmaceutically acceptable salt or an in vivo hydrolysable ester thereof wherein Y is selected from phenyl and a five- or six-membered aromatic ring containing one or more heteroatoms independently selected from N, O, and S.

11. A compound as claimed in claim 10 or a pharmaceutically acceptable salt or an in vivo hydrolysable ester thereof, wherein Y is selected from phenyl, pyridyl, pyrimidinyl, or pyrazinyl.

12. A compound as claimed in claim 1 or claim 2 or a pharmaceutically acceptable salt or an in vivo hydrolysable ester thereof, wherein Y is not substituted or Y is substituted by at least one group independently selected from halogen, CF₃, and MeO.

13. A compound as claimed in claim 12 or a pharmaceutically acceptable salt or an in vivo hydrolysable ester thereof, wherein Y is not substituted or Y is substituted by at least one halogen group.

14 A compound as claimed in claim 1 or a pharmaceutically acceptable salt or an in vivo hydrolysable ester thereof, wherein the compound is selected from Hydroxy[4-pyrimidin-2-yl-1-({[4-(4-thien-3-ylphenyl)piperazin-1-yl]sulfonyl}methyl)butyl]formamide, 1-[({4-[5-(4-fluorophenyl)pyridin-2-yl]piperazin-1-yl} sulfonyl)methyl]-4-pyrimidin-2-ylbutyl(hydroxy)formamide, 1-[({4-[5-(4-chlorophenyl)pyridin-2-yl]piperazin-1-yl} sulfonyl)methyl]-4-pyrimidin-2-ylbutyl(hydroxy)formamide, 1-[({4-[5-(3-furyl)pyridin-2-yl]piperazin-1-yl} sulfonyl)methyl]-4-pyrimidin-2-ylbutyl(hydroxy)formamide, 1-({[4-(2,3′-bipyridin-6′-yl)piperazin-1-yl]sulfonyl} methyl)-4-pyrimidin-2-ylbutyl(hydroxy)formamide, hydroxy[4-pyrimidin-2-yl-1-({[4-(5-thien-2-ylpyridin-2-yl)piperazin-1-yl]sulfonyl}methyl)butyl]formamide, 1-[({4-[5-(2-furyl)pyridin-2-yl]piperazin-1-yl} sulfonyl)methyl]-4-pyrimidin-2-ylbutyl(hydroxy)formamide, 1-[({4-[5-(4-fluorophenyl)pyrazin-2-yl]piperazin-1-yl} sulfonyl)methyl]-4-pyrimidin-2-ylbutyl(hydroxy)formamide, hydroxy[1-({[4-(5-phenylpyrazin-2-yl)piperazin-1-yl]sulfonyl} methyl)-4-pyrimidin-2-ylbutyl]formamide, 1-[({4-[5-(3-furyl)pyridin-2-yl]piperazin-1-yl} sulfonyl)methyl]-4-pyrimidin-2-ylbutyl(hydroxy)formamide, 1-[({4-[5-(4-fluorophenyl)pyrimidin-2-yl]piperazin-1-yl} sulfonyl)methyl]-3-pyrimidin-2-ylpropyl(hydroxy)formamide, (1S)-1-[({4-[5-(4-fluorophenyl)pyrimidin-2-yl]piperazin-1-yl} sulfonyl)methyl]-3-pyrimidin-2-ylpropyl(hydroxy)formamide, (1S)-1-[({4-[5-(4-fluorophenyl)pyrimidin-2-yl]piperazin-1-yl} sulfonyl)methyl]-4-pyrimidin-2-ylbutyl(hydroxy)formamide, 1-[({4-[5-(2-chloro-4-fluorophenyl)pyrimid-2-yl]piperazin-1-yl} sulfonyl)methyl]-4-pyrimidin-2-ylbutyl(hydroxy)formamide, (1R or 1S)-1-[({4-[5-(2,4-difluorophenyl)pyrimid-2-yl]piperazin-1-yl} sulfonyl)methyl]-4-pyrimidin-2-ylbutyl(hydroxy)formamide, 1-[({4-[5-(2-Fluorophenyl) pyrimidin-2-yl]piperazin-1-yl} sulfonyl)methyl]-3-pyrimidin-2-ylpropyl(hydroxy)formamide, hydroxy[1-({[4-(5-pyridin-2-ylpyrimidin-2-yl)piperazin-1-yl]sulfonyl}methyl)-4-pyrimidin-2-ylbutyl]formamide, and 3-(5-fluoropyrimidin-2-yl)-1-({[4-(5-pyridin-2-ylpyrimidin-2-yl)piperazin-1-yl]sulfonyl} methyl)propyl(hydroxy) formamide.

15. A pharmaceutical composition, which comprises a pharmaceutically acceptable carrier and a compound of claim 1 or claim 2 or a pharmaceutically acceptable salt or an in vivo hydrolysable ester thereof.

16. A method for treating a human or animal, comprising administering to the human or animal a therapeutic amount of a compound of claim 1 or claim 2 or a pharmaceutically acceptable salt or in vivo hydrolysable ester thereof.

17. A method of treating a metalloproteinase mediated disease or condition which comprises, administering to a warm-blooded animal a therapeutically effective amount of a compound of claim 1 or claim 2 or a pharmaceutically acceptable salt or in vivo hydrolysable ester thereof.

18. A method of treating a metalloproteinase mediated disease condition as claimed in claim 17, wherein the metalloproteinase is MMP13.

19. A method for treating a disease or condition mediated by one or more metalloproteinase enzymes, comprising administering to a patient in need thereof a therapeutically effective amount of a compound of claim 1 or claim 2 or a pharmaceutically acceptable salt or in vivo hydrolysable precursor thereof.

20. A method for treating arthritis, comprising administering to a patient in need thereof a therapeutically effective amount of a compound of claim 1 or claim 2 or a pharmaceutically acceptable salt or in vivo hydrolysable precursor thereof.
